(19) United States
(12) Patent Application Publication
(10) Pub. No.: US 2005/0123911 A1
(43) Pub. Date: Jun. 9, 2005

(54) Method of mass spectrometry

(76) Inventor: Stephen Carl McKeown, Harlow (GB)

Correspondence Address: DAVID J LEVY, CORPORATE INTELLECTUAL PROPERTY;GLAXOSMITHKLINE, FIVE MOORE DR., PO BOX 13398, RESEARCH TRIANGLE PARK, NC, 27709-3398, US

(21) Appl. No.: 10/481,936

(22) PCT Filed: Jun. 25, 2002
(86) PCT No.: PCT/GB02/02921

(30) Foreign Application Priority Data
Jun. 26, 2001 (GB) 0115581.1

(51) Int. Cl. C12Q 1/68; G01N 33/53; G01N 33/537; G01N 33/543; C12Q 1/37

(52) U.S. Cl. 435/6; 435/7.92; 435/23

(57) ABSTRACT

A method of analyzing the cleavage of a polymer wherein the polymer is differentially labeled with an isotope and the cleaved fragments of the polymer are analysed by mass spectrometry is disclosed. The method is particularly useful for analyzing the cleavage of proteins and more particularly for determining substrate specificity for enzymes such as proteases.

### FIELD OF THE INVENTION

The present invention relates to methods of analysing cleavage of a polymer wherein the polymer is differentially labelled with an isotope and the cleaved fragments of the polymer are analysed by mass spectrometry. In particular it relates to methods of determining a cleavage site in the polymer, screening methods and methods of obtaining information about the structure and sequence of cleaved polymers.

### BACKGROUND OF THE INVENTION

Chemical labels are widely used in chemical analysis. Among the types of molecules used are radioactive atoms, fluorescent reagents, luminescent reagents, metal-containing compounds, electron absorbing substances and light absorbing compounds. Such chemical labels may be covalently attached to the target to enable the substance to be detected. However, chemical moeities present on the target surface may interfere with the detection of the label. Such labels have been used in methods to identify the substrates of enzymes and their cleavage sites. However, such labels may have further limitations since the labels themselves can have an influence on the cleavage kinetics. There is therefore a need for a method of labelling potential substrates for such cleavage analysis which involves minimal perturbation of the structure of the substrate, presenting the substrate molecule almost indistinguishable from its unlabelled state.

In recent years, the approach of mass spectrometry has been used in the identification of proteins. Proteins are usually identified by the best match between the measured and calculated m/z (mass to charge ratio) values of the peptides. This approach however, encounters a number of problems such as low sample availablility, the presence of background or artifact ions and postsynthetic modification of proteins. Ultrahigh accuracy instruments may be required to determine absolute m/z values.

Recently, a more accurate approach has been reported wherein stable isotopic labels are incorporated into proteins. Uniformly ¹⁵N-labelled proteins have been generated for improved accuracy of mass measurements by mass spectrometry (Jensen et al Anal Chem 71: 2076-2084 (1999)). Specific amino acids have been labelled with stable isotopes for incorporation into proteins so that these proteins may be distinguished from other molecules in a mass spectrum (Chen et al Anal Chem 72: 1134-1143 (2000)). ¹⁸O labels have been used in the identification of C terminal peptides of proteins (Kosaka et al Anal Chem 72:1179-1185 (2000)).

### SUMMARY OF THE INVENTION

The present invention provides a method of analysing cleavage of a polymer, the method comprising:
(a) providing a sample of said polymer, a portion of said polymer molecules having been labelled at a position on one side of a potential cleavage site with a first isotopic label and a portion of said polymer molecules having been labelled at a position on the opposite side of the potential cleavage site with a second isotopic label,
(b) incubating said sample under conditions suitable for cleavage at said potential cleavage site, and
(c) analysing the mass(es) of any cleaved fragments by mass spectrometry and thereby determining whether and/or where cleavage has taken place.

Preferably, the mass change caused by the presence of the first isotopic label compared with the mass in the presence of the normal element(s) at that position is different to the mass change caused by the presence of the second isotopic label compared with the mass in the presence of the normal element(s) at that position,

The present invention further provides a method of screening a library of polymers for cleavage, the method comprising:
(a) providing a library comprising two or more polymers labelled as described in claim 1,
(b) incubating said library of polymers under conditions suitable for cleavage, and
(c) analysing the mass(es) of any cleaved fragments by mass spectrometry and thereby determining whether and/or where cleavage has taken place.

The sample may be incubated with an enzyme under conditions suitable for the cleavage of a substrate.

In a further aspect of the invention, the method further comprises the step of quantifying the amount of the polymer or cleaved fragment(s) present after cleavage.

In another aspect of the invention, said polymer further comprises a detectable non-isotopic label on one side of the potential cleavage site and an inhibitor for said label on the opposite side of the potential cleavage site, and the method comprises the additional step of detecting said detectable non-isotopic label, wherein the detection of said label indicates that said polymer has been cleaved.

The methods of the present invention may further comprise determining the structure or sequence of said polymer or of one or both of the cleaved fragments, optionally by further cleaving these fragments and determining the masses of these further fragments using mass spectrometry.

The present invention further comprises a kit for screening a library of polymers for cleavage, comprising two or more differentially isotopically labelled polymers as described above. The invention also comprises a kit for the preparation of such a polymer, comprising a set of chemical monomers which comprises monomers which have been isotopically labelled and monomers which have no isotopic label, in a form suitable for polymer synthesis.

### BRIEF DESCRIPTION OF THE DRAWINGS

FIG. 1: Sample mass spectra for uncleaved (A-B) and cleaved (A, B) peptides labelled with either: [symbol] =D2 or H2 Gly, or [symbol] =D3 or H3 acetate.

FIG. 2: Mass Spectra before and after cleavage of [symbol] -Gly-Pro-Arg-Ala-Ala-Ala-Gly- [symbol] with trypsin.

FIG. 3: Mass spectrum of peptide 4.

FIG. 4: Mass spectrum of peptide 4 following treatment with trypsin.

FIGS. 5a and b: Mass spectra of peptide 4 following treatment with NEP.

FIG. 6: Summary of sample method used to screen peptides (A-B) for cleavage by an enzyme, isolation of peptide fragment A and any remaining peptide A-B and identification of their peptide sequences. MS1 and MS2=isotopic labels.

FIG. 7: Determination of optimal ratio of fluorescent group (F) to quencher (Q).

FIG. 8: Mass spectrum analysis for resin 16.

FIG. 9: Mass spectrum analysis for resin 17.

FIG. 10: Mass spectrum analysis of resin 15 following treatment with trypsin.

FIG. 11: Strategy for synthesis of a library of differentially isotopically labelled compounds.

FIG. 12: Mass spectra of compound 7₍₂₎ (bj00080/2), compound 7₍₁₇₎ (bj00083/5), compound 7₍₂₄₎ (bj00014/9), and compound 7₍₃₁₎ (bj00014/13) respectively.

FIG. 13: A Mass spectrum following incubation of compound 7₍₂₎ with trypsin: cleaved sequence H2N-Ala-Asn-Ile-Asp-Phe-Ala-Lys(ac)-NH2 (819/822). B, C: Mass spectra of compound 7₍₁₇₎ with trypsin: B: cleaved sequence Ac-Gly-Ala-Ala-Phe-Lys-Arg-OH (691/693), C: cleaved sequence Ac-Gly-Ala-Ala-Phe-Lys-OH.

FIG. 14: Mass spectra of compounds 8₍₁₋₄₎.

FIG. 15: Mass spectra of compounds 8₍₅₋₈₎.

FIG. 16: Mass spectra of compounds 8₍₁₋₄₎ following treatment with trypsin.

FIG. 17: Mass spectra of compounds 8₍₅₋₈₎ following treatment with trypsin.

FIG. 18: Amino acid sequencing via mass spectrometry on peptide fragment differentially labelled with a stable isotope ( [symbol] ) at one terminal. Mass spectra show sample pattern produced by cleavage of amino acids from either the C or N-terminal of the peptide.

### DETAILED DESCRIPTION OF THE INVENTION

A series of methods for analysing the cleavage of a polymer have been developed based on the differential isotopic labeling of the polymer. These methods may be useful in discovering new or improved synthetic substrates for for both known and unknown enzymes, for example novel enzymes identified from the human genome. The methods may be used to identify an event of cleavage, the site of cleavage and additionally the sequence origin.

The methods of the present invention may also, for example, be used in screening methods to identify new substrates for enzymes, in positional peptide scanning libraries, in in vitro/ex-vivo/in vitro peptide tracking, in assaying methods, for oligonucleotide or peptide sequencing or in the measurement of differential protein expression.

Differentially Isotopically Labelled Polymers

The present invention is based on the use of an isotopic label in the analysis of cleavage of a polymer.

The term “polymer” refers to any molecule made up of discrete “components”. A suitable polymer for use in a method of the present invention may be any polymer which can be cleaved. A suitable polymer may, for example, be a biological polymer such as a peptide, polypeptide, protein, polynucleic acid (e.g. DNA, RNA or LNA/DNA/RNA hybrid), lipid or carbohydrate. In a preferred aspect of the invention, the polymer comprises a peptide or protein.

The term “component(s)” refers to monomers which make up the polymer, e.g. amino acids (for proteins), nucleic acids (for DNA or RNA), sugars (for complex carbohydrates) and fatty acids (for lipids). A polymer will comprise 3 or more such components. A polymer may comprise, for example, 3, 5, 10, 20, 50, 100, 500 or more such components. A suitable polymer may be an oligomer, such as an oligonucleotide or oligopeptide. Preferably, the polymer is a linear polymer.

For use in the methods of the present invention, a sample of a polymer is differentially isotopically labelled. A sample of the polymer is partially labelled on either side of a potential cleavage site with a first and a second isotopic label such that a portion of said polymer molecules are labelled at a position on one side of the potential cleavage site with a first isotopic label and a portion of said polymer molecules are labelled at a position on the opposite side of the potential cleavage site with a second isotopic label. The two labelled portions are not mutually exclusive, i.e. some polymer molecules may fall within both portions and may therefore comprise both a first and a second isotopic label.

Such a sample may therefore comprise a mixture of polymer molecules which are unlabelled, polymer molecules which have only the first isotopic label, polymer molecules which have only the second isotopic label and polymer molecules which have both isotopic labels. Other suitable samples may comprise a mixture of polymer molecules which are unlabelled and polymer molecules which have both isotopic labels or a mixture of polymer molecules which have only the first isotopic label and polymer molecules which have only the second isotopic label. A suitable sample may comprise any combination of unlabelled, singly-labelled or doubly-labelled polymer molecules as long as the sample comprises both the first and second isotopic label.

By partially labelling in this way at each of these sites, each cleaved fragment will show a characteristic mass spectrum of two peaks representing the different isotopes present. The precise pattern of the mass spectrum will depend upon the relative amounts of the individual isotopes in the sample. Two fragments formed by cleavage of a polymer will therefore show a distinctive pattern of two pairs of mass peaks as described below.

A suitable sample may be 50% differentially labelled with isotopic labels on both sides of the potential cleavage site. That is, 50% of the polymer molecules in a sample are labelled at a position on one side of the potential cleavage site with the first isotopic label, and 50% of the polymer molecules in the sample are labelled at a position on the opposite side of the potential cleavage site with the second isotopic label. Such a sample may therefore comprise only polymer molecules which have either the first or the second isotopic label, or may comprise any combination of unlabelled, singly-labelled and doubly-labelled polymer molecules as described above. If a given atom is labelled such that 50% of the atoms are of one isotopic form and 50% are of another isotopic form, the mass spectrum of the mixture will show a characteristic doublet in which the peaks are of approximately equal height.

A suitable sample comprises polymers having isotopic labels on either side of a potential cleavage site. For use in a method of the present invention, a polymer will be labelled at specific positions on each side of the cleavage site. Preferably, an isotopic label will be located on a single monomer within the polymer. A polymer may be labelled, for example, by substitution of a monomer by a labelled monomer, by addition of a labelled monomer or by addition of labelled moeities within or at an end of the polymer molecule. For example, if the polymer is a peptide, it may be labelled by substituting an amino acid by a labelled amino acid, by addition of a labelled amino acid to the polymer or by addition of a labelled moeity to the peptide, such as by acetylation, hydroxylation, carboxylation or phosphorylation.

Suitable positions for labelling may be determined for individual polymers based on, for example, the location of potential cleavage sites or the particular isotopic labels used. If the cleavage site is known, the isotopic labels can be positioned in the polymer on the appropriate sides of that cleavage site. For example, each isotopic label may be present at a terminal end of the molecule, adjacent to the cleavage site itself or at any intermediate location. If the potential cleavage site is unknown, the isotopic labels may, for example, be positioned towards the terminal ends of the polymer. If the polymer is a peptide, the isotopic labels may therefore be present in the region of the terminal amino acids, such as in the terminal five amino acids, or in moeties attached to these. The isotopic labels may preferably be present in the terminal monomers themselves, e.g. if the polymer is a peptide, the isotopic labels may be present in the terminal amino acids. Alternatively, the isotopic labels may be present in moeties attached to the polymer, preferably to the terminal monomer of the polymer. For example, the isotopic label may be introduced by acetylation at the amino terminus of a peptide.

A suitable isotopic label may comprise one or more chemical isotopes of one or more atoms present within the unlabelled polymer. An isotope of an element will have an identical number of protons and electrons, but a different number of neutrons, and therefore a different molecular weight. For example, an isotope may have one additional neutron and therefore have an increased molecular weight by one mass unit. Preferably each isotope is substituted for the normal atom within the polymer. For example, ¹H may be substituted by ²H (deuterium), ¹²C by ¹³C, ¹⁶O by ¹⁸O, ¹⁴N by ¹⁵N and/or ⁷⁹Br by ⁸¹Br. One or more different isotopes may be substituted into a polymer. The same isotope may be substituted one or more times into a polymer. A suitable isotope will have a half-life which is sufficiently long to minimise the likelihood of isotopic decay during the method. Preferably the half-life is greater than the amount of time between incorporation of the isotope into the monomer and the analysis by mass spectrometry of any cleaved fragments. Preferably the isotope has a half life of at least one day, more preferably at least one week. Most preferably the isotope is a stable isotope such as an isotope which has a half life of many years or many hundreds or thousands of years.

The first and second isotopic labels may be the same or different and may comprise the same chemical isotopes or different chemical isotopes.

The first and second isotopic labels may lead to the same mass change in the labelled polymer. For example, two hydrogen atoms may be substituted by two deuterium atoms on one side of a potential cleavage site leading to a mass change in the polymer of two atomic mass units. On the other side of the potential cleavage site two more hydrogen atoms may be substituted by two deuterium atoms, also leading to an increase in mass of two atomic mass units. As explained below, as each position will only be partially labelled in a sample of polymer, this will lead to a distinctive mass spectrum for both the polymer and its cleaved fragments.

In a preferred aspect of the present invention, the mass change caused in the polymer by the presence of the first isotopic label compared with the mass in the presence of the normal element(s) at that position is different to the mass change caused in the polymer by the presence of the second isotopic label compared with the mass in the presence of the normal element(s) at that position. For example, two hydrogen atoms may be substituted by two deuterium atoms on one side of the cleavage site and three hydrogen atoms substituted by three deuterium atoms on the other side of the cleavage site. Alternatively, one ¹²C atom may be substituted by a ¹³C atom on one side of the cleavage site and two hydrogen atoms substituted by two deuterium atoms on the opposite side of the cleavage site.

Where two or more cleavage sites exist in the polymer, additional isotopic labels may be incorporated. Sufficient isotopic labels may be incorporated such that one is present in each cleaved fragment. Each isotopic label should cause a different mass change in the polymer when compared with the mass in the presence of the normal element(s) at that position.

The synthesis of isotopically labelled polymers and the introduction of isotopic labels to polymers may proceed by way of common organic and biochemical reactions and techniques. A polymer may be labelled in a number of different ways, at one or more different molecular positions or with different isotopes, either singly or in combination. Such methods are well understood by persons skilled in the art, and include techniques such as those disclosed in Ott, D. G., Syntheses with Stable Isotopes of Carbon Nitrogen and Oxygen, John Wiley & Sons, 1981, the disclosure of which is included herein in its entirety by way of reference. An isotopic label may be incorporated into a monomer which is then introduced into the polymer molecule. For example a particular labelled amino acid may be incorporated into a protein during cell growth or in an in vitro transcription/translation system as described in Yabuki et al (J Biomol NMR 11:295-306 (1998)).

In one embodiment the polymer(s) may be attached to a solid support. The solid support may take the form of, for example, beads, a solid surface, a solid substrate, particles, pellets, discs, capillaries, hollow fibres, needles, solid fibres, organic or inorganic gels or insoluble inorganic particles such as particles formed from fullerenes. Beads may be polymeric beads such as cellulose beads or resin beads. Resin beads may be produced from functionalised polymer resins such as polystyrene resins, polyacrylamide resins and dimethylacrylamide resins.

The polymer may be attached to a solid support by a linker molecule. Preferably the linker molecule is a cleavable linker molecule. Preferably the linker molecule is selectively cleavable, for example chemically cleavable, but is not cleaved by the method (e.g. enzyme) used in the screening assay.

Methods of Analysing Cleavage of a Polymer

The methods of the present invention comprise the incubation of polymers under conditions suitable for cleavage.

The method of cleavage may be any method which leads to the cleavage of a polymer e.g. mechanical, chemical or enzymatic cleavage. In a preferred aspect of the invention, the polymer is incubated with an enzyme. For example, if the polymer is a polypeptide or protein, the enzyme may be a protease, if the polymer is a polynucleotide, the enzyme may be an endonuclease or exonuclease, if the polymer is a carbohydrate, the enzyme may be an amylase or glucanase and if the polymer is a lipid the enzyme may be a lipase. The polymer is incubated with an enzyme under conditions suitable for the cleavage of a substrate. Suitable incubation conditions will vary with the cleavage method, such as with the enzyme used, and may be easily determined by the person skilled in the art.

The mass of the cleaved fragments may be determined by any method known in the art. The mass should be measured sufficiently accurately that a molecular mass of each fragment may be determined. Preferably the mass should be measured sufficiently accurately that the mass change caused by the isotopic labels may be detected.

In a preferred aspect, the isotopic label(s) are detected by a method of mass spectrometry. Suitable methods of mass spectrometry include matrix-assisted laser-desorption ionization mass spectrometry, direct laser-desorption ionization mass spectrometry, electrospray ionization mass spectrometry, secondary neutral mass spectrometry and secondary ion mass spectrometry.

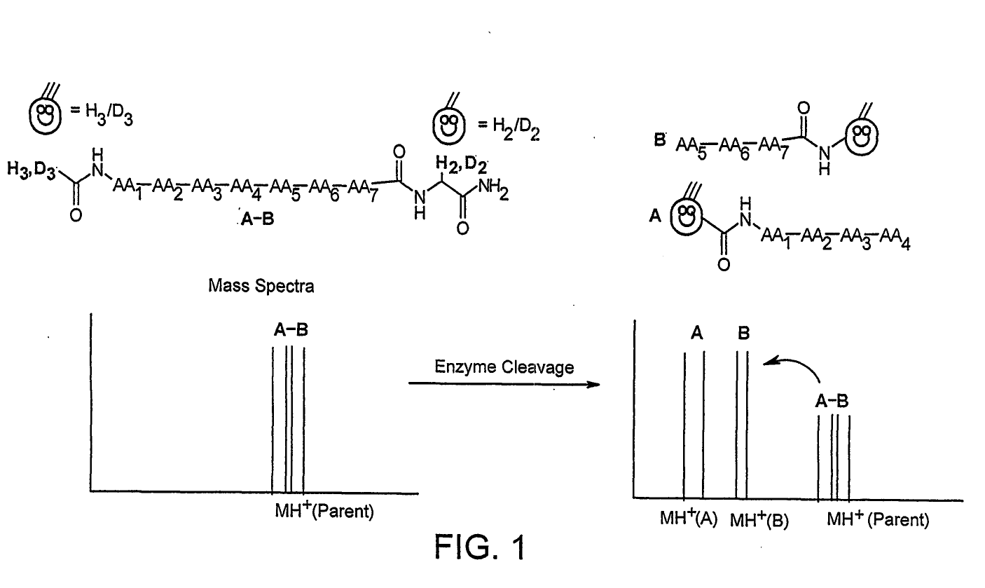

The cleaved fragments will show a distinctive pattern of two pairs of mass peaks (see FIG. 1). Each fragment will be present in labelled and unlabelled form, each being seen as a separate peak. A fragment comprising an isotopic label will show a peak which is shifted with respect to the equivalent unlabelled fragment because of the different molecular weight of the isotope(s) compared with the normal element(s). This distinctive pattern allows easy determination of whether cleavage has occurred. The presence of one or two such pairs of peaks indicates that the polymer has been cleaved. The mass of each fragment may be determined from such a spectrum. This distinctive pattern of pairs of peaks representing labelled cleaved fragments facilitates the identification of the molecules of interest without the need to extract and purify them from the sample or reaction mixture.

In a preferred aspect the sample of polymer cleaved comprises unlabelled polymer molecules, polymer molecules comprising the first isotopic label only, polymer molecules comprising the second isotopic label only and polymer molecules comprising both the first and second isotopic labels.

If the first and second isotopic labels are the same, or are different but lead to the same mass change in the polymer, this will lead to a characteristic mass spectrum of three peaks. One peak corresponds to the unlabelled polymer, one to polymer containing only one isotopic label and the third peak corresponds to polymer containing both the first and second labels. As both labels lead to the same mass change in the polymer, polymer containing either the first or the second label will have the same molecular weight and these two types of molecule will be seen as a single peak on a mass spectrum.

If the first and second isotopic labels lead to a different mass change in the polymer, analysis of an uncleaved sample will show a characteristic mass spectrum of four peaks (see FIG. 1). One peak corresponds to the unlabelled polymer, one to polymer comprising the first isotopic label but not the second (shifted with respect to the unlabelled polymer because of the different molecular weight of the isotope(s) compared with the normal element(s)), a third peak will correspond to polymer comprising the second isotopic label but not the first, and a final peak will correspond to polymer comprising both isotopic labels (shifted by the different molecular weights of both isotopic labels). As the first and second isotopic labels lead to a different mass change in the polymer, polymer molecules having only the first label will have a different molecular weight to polymer molecules having only the second label. Two separate peaks corresponding to polymer containing each of these labels should therefore be seen on a mass spectrum. The precise pattern will depend upon the relative amounts of the different isotopes.

In either of these cases, the distinction between the three or four peaks of the uncleaved polymer and the two pairs of peaks of the cleaved fragments make it possible to readily determine from a mass spectrum whether cleavage has taken place.

The methods of the present invention may be used to determine the specific location of a cleavage site within a polymer. In the embodiment wherein the first and second isotopic labels cause different mass changes when compared with the mass of the polymer in the presence of the normal isotope(s) at their positions, it can be determined from the spectrum which pair of peaks corresponds to which cleaved fragment. Each pair of peaks will show a split equivalent to the difference in mass between a labelled and unlabelled fragment (see FIG. 1). The individual cleaved fragments may therefore be identified from such a mass spectrum, their masses determined, the sequence established and the exact location of the cleavage site within the polymer may therefore be inferred.

If the polymer has been cleaved once, at a position between the two isotopic labels, the sum of the molecular weights of the cleaved fragments should be equal to the molecular weight of the uncleaved polymer.

If the sum of the molecular weights of the cleaved fragments is less than the molecular weight of the uncleaved polymer, this indicates that cleavage has also occurred elsewhere within the polymer. The location of other such cleavage sites may be determined by incorporating additional isotopic labels in the polymer as described above and analysing the cleaved fragments as described above; by determining the structure or sequence of the isotopically labelled fragments as described below; or by a combination of these two methods.

Data obtained by mass spectrometry may be analysed by any suitable software. For example, peak split recognition software (‘cluster analysis’) is available from Micromass© Ltd and has to date been used for analytical construct and bead decoding technologies (McKeown S. C., Watson, S., Carr R., Marshall, Tetrahedron Lett (1999) 40: 2407; Lane S. J., Pipe A. J., Rapid Communications in Mass Spectrometry (2000) 14: 782-793). Such software simplifies the data interpretation by filtering out specific ‘peak splits’. Such software could also be used to automate the interpretation of the mass spectra obtained using the methods of the present invention.

Quantification

Methods of the invention may also be used as part of an assaying technique based of mass spectrometry, for example by quantification of the amount of cleavage or the amount of the polymer or cleaved fragment(s) in a sample. For example, the amount of cleavage which has occurred following any of the methods of the invention described herein may be quantified.

The polymer may further contain a moiety which aids quantification. Suitable moieties would be well known in the art. Methods of quantification (and suitable moieties for use in such methods) include, for example, UV, Fluorescent and Visible Spectroscopy (e.g. using p-nitrophenol,dansyl, aminomethylcoumarin), Inductively coupled Plasma Mass Spectrometry (ICP-MS), (e.g. using Chlorine, Bromine, Sulphur, Phosphorus), NMR, Weighing, Acoustic Photo Spectroscopy, Mass Spectrometry, Raman Spectroscopy, Atomic Absorption, Elemental Analysis, Electrolysis, Circular Dichroism, Elisa, ESR (using Electron Spin Resonance). 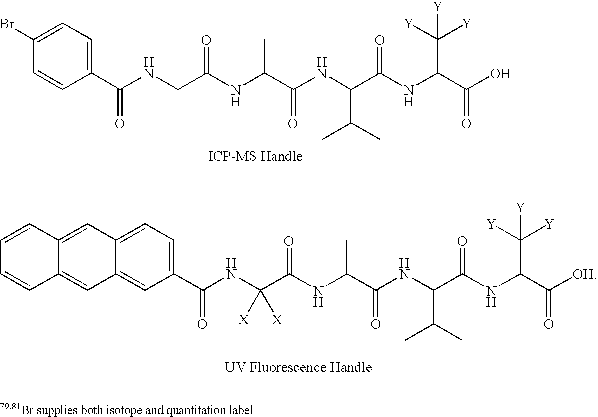
The polymer may also contain combined fluorescent-quench pairs (e.g. FRET pairs such as FAM/TAMRA):

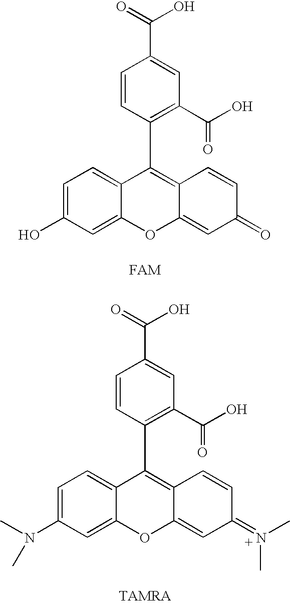

Additionally the polymer may contain a sensitising group to enhance detection by the Mass Spectrometer. For example to enhance a molecule to ESI-MS (positive mode) an amino group can be incorporated. For example:

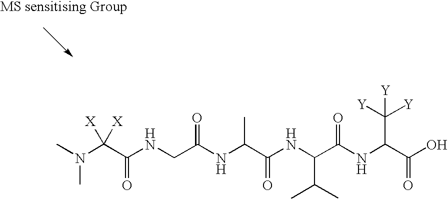

The polymer may additionally or alternatively comprise a moeity which may be used in the separation of the polymer or its cleaved fragments from other material. A suitable moeity may be one of a specific binding pair such as biotin. A suitable moeity may be removable from the polymer molecule, for example by specific cleavage. A moeity may be present on one side of the potential cleavage site or on both sides of the potential cleavage site. The presence of such a moeity would allow for the polymer or its fragments to be more easily separated from the cleavage mixture, for example, using the other of the specific binding pair to capture polymer or cleaved fragments that comprise S the moeity. The other of the specific binding pair may be bound to a solid support and may capture molecules comprising the moeity when these are incubated with or passed over the solid support. These binding pair complexes including the polymer or cleaved fragment may then be removed from the solid support.

More than one such moeity may be attached to a polymer molecule. The same moeity may be attached to the polymer molecule at more than one location. If the moeity is present on both sides of the potential cleavage site, it may be used to separate uncleaved polymer and any cleaved fragments from the cleavage mixture, for example from any enzymes or chemicals which are used to induce cleavage. Alternatively, two or more different moeities may be attached to the polymer molecule. For example, a polymer for use in a method of the present invention may comprise a different moeity on each side of the potential cleavage site. Each cleaved fragment may then be captured separately, for example by simultaneously or separately using the others of the specific binding pairs to capture each cleaved fragment. In this way the cleaved fragments may be separated from each other.

Screening Methods

The methods of the invention may be used to screen a library of polymers for those polymers which are cleaved by a particular method, for example by a particular enzyme. The methods of the invention may, for example, be used to identify substrates for a particular enzyme, e.g. a protease. Potential substrates may be prepared by introducing isotopic labels as described above. The substrates may be prepared as single compounds or large libraries, e.g. 10¹⁰. These individual or mixed substrates may then be incubated with one or more enzymes. The resulting fragments may then be analysed by mass spectrometry and the cleaved substrates identified.

Alternatively, the methods may be used to screen a library of enzymes for the ability to cleave a particular substrate.

The methods of the present invention may be used to monitor the cleavage of polypeptides or polynucleotides, for example, polypeptides or polynucleotides which vary by one or more amino acids or nucleotides respectively at a site which effects the ability of the enzyme to cleave the substrate. The site may or may not be part of the recognition sequence for the enzyme. For example, for a polynucleotide, the cleavage site may be selected to be related to a specific sequence or nucleotide polymorphism, for example a single nucleotide polymorphism (SNP). The polypeptides or polynucleotides are differentially isotopically labelled as described above. They may then be treated to induce cleavage, preferably specific cleavage at the site of the variation e.g. by introducing an enzyme such as a protease (for polypeptides) or an endonuclease or exonuclease (for polynucleotides). Preferably the ability of the enzyme to cleave the substrate will depend on the sequence or nucleotide which varies between the substrates. The fragments produced may be analysed by mass spectrometry. The methods of the present invention may therefore be used to detect specific variants in the polypeptide(s) or polynucleotide(s). Such methods may further be linked to a molecular diagnostic assay technology. Such an assay may involve, for example, target amplification (e.g. PCR) or signal amplification (e.g. a branched DNA assay).

For any of the above screening approaches, the cleavage sites in such polymers may be determined by the methods described above, and the sequences of such molecules may be determined by methods as described below.

A suitable polymer for use in a screening method of the invention will comprise any differentially isotopically labelled polymer as described above. A suitable polymer may further comprise a detectable label on one side of the cleavage site and an inhibitor for that label on the opposite side of the cleavage site.

A suitable polymer may be synthesised by methods known in the art. For example, it may be produced by attaching a detectable label and an inhibitor to any of the polymers described above.

A suitable detectable label may be any label which may be reliably detected in a screening method. A detectable label may be a moiety which aids quantification or separation as described above. Preferably the detectable label is a chemical label. For example, suitable chemical labels may include radioactive atoms, fluorescent reagents, chromophores, luminescent reagents, metal-containing compounds, electron absorbing substances and light absorbing compounds. A suitable inhibitor will be one which prevents the detection of the detectable label. Preferably the inhibitor will inhibit the detection of the detectable label while in close proximity to the label (i.e. when the polymer is intact) but will no longer inhibit the detection of the label when the polymer is cleaved. In a preferred aspect, the detectable marker is a fluorophore and the inhibitor is a quencher for that fluorophore.

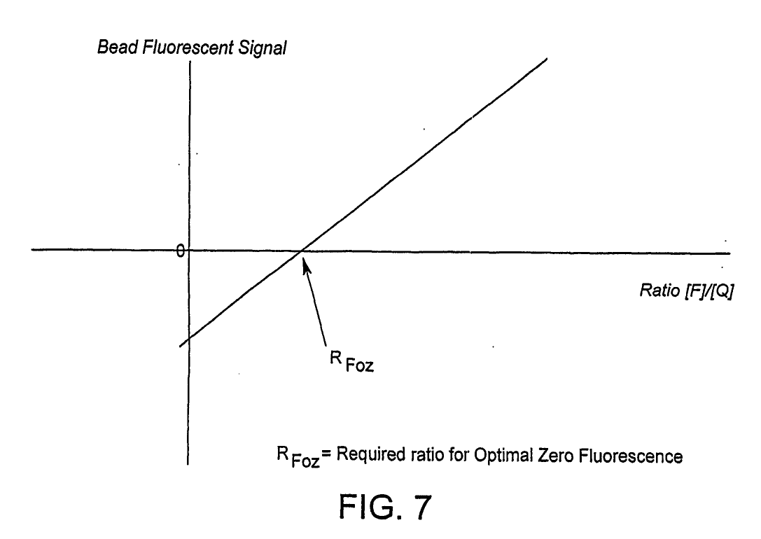

Preferably, the ratio of detectable marker to inhibitor is optimised such that in the absence of cleavage the marker is not detectable, but on cleavage the marker becomes detectable (see FIG. 7). An appropriate ratio of detectable marker to inhibitor will depend on the particular marker and inhibitor used and the method of detection. An appropriate ratio for a particular combination of label and inhibitor may be determined by the skilled person using methods known in the art.

A suitable polymer may be used in a screening method comprising the steps of incubating under conditions suitable for cleavage and screening for the detectability of the label. If the polymer is not cleaved, the inhibitor should prevent the detectability of the label. If the polymer is cleaved by the enzyme, the inhibitor will be separated from the label and the label should be detectable. The cleaved fragment comprising the label may then be identified and isolated. The cleaved fragment may be analysed using mass spectrometry as described above and its sequence or structure may be determined as described below.

In one embodiment the polymer is attached to a solid support. Any suitable solid support may be used, for example those solid supports described above. The polymer may be attached to the solid support via a linker molecule. Preferably the linker molecule is a cleavable linker molecule. Preferably the linker molecule is selectively cleavable, for example chemically cleavable, but is not cleaved by the method (e.g. enzyme) used in the screening assay.

Preferably the fragment which is cleaved from the support-linker-polymer construct during the assay comprises the inhibitor. The detectable marker may be attached to the polymer, between the polymer and any linker molecule, directly to any linker molecule, between a linker molecule and the solid support or it may be attached directly to the solid support. One or more polymers may be attached to a solid support.

In a preferred aspect the polymer is attached to a resin bead. For example, a library of polymers may be synthesised on resin beads that incorporate fluorescent and quenching groups. A number of molecules of each polymer may be synthesised on an individual bead. The library may be designed such that after polymer cleavage, the resulting bead emits a fluorescent signal on irradiation (see calculation of R_{foz} below). This fluorescence signals the event of cleavage. The recognisable bead can be selected and further analysed. The cleaved (isotopically labelled) polymer, along with any remaining, uncleaved polymer, may be then released from a bead by specific cleavage of the linker. The released polymer(s) may then, for example, be subjected either to mass measurement, for example by mass spectrometry, to confirm the event of cleavage, to identify the site of hydrolysis as described above, or to further fragmentation to allow identification of their sequences as described below.

Substrate Optimisation

The methods of the present invention may be utilised in a number of methods to determine the optimal polymer substrate for a particular enzyme or other cleavage method.

The methods of the present invention may be used in positional scanning libraries, for example in positional scanning peptide libraries. A number of polymers (e.g. peptides) may be synthesised which vary by a single monomer (e.g. a single amino acid). These polymers are differentially isotopically labelled as described above. They may then be treated to induce cleavage, e.g. by introducing an enzyme, and the fragments produced analysed by mass spectrometry. The cleavage conditions may be varied and the data obtained analysed to determine the optimal monomers required in the polymer substrate for that cleavage method (e.g. enzyme).

A polymer of a given length may be systematically shortened to investigate the contribution of individual monomers to cleavage. Such a method may be used to find the shortest sequence of monomers which would still allow for a cleavage even to occur. Differentially isotopically labelled polymers as described above may be used in such a method. In such methods, the truncated permutations may be simultaneously synthesised producing a mixture of differentially isotopically labelled sequences. The ability to identify labelled fragments by their label and length as described above, rather than identifying the starting polymer which has been cleaved, makes it possible to perform cleavage on such a mixture of polymers together.

Tracking and Diagnostics

Differentially isotopically labelled polymers as described above may also be used in tracking experiments. For example, biological polymers such as peptides may be differentially isotopically labelled as described above and the course of such polymers in a biological system tracked by analysis using mass spectrometry.

For example, such labelled peptides may be used for in vitro/ex vivo/in vitro peptide tracking. Biological or chemical degradation of such polymers may also be tracked by identifying the appearance of pairs of peaks by mass spectrometry indicating that the labelled molecule has been cleaved.

Such methods also find applications in diagnostics. A polymer which has been differentially isotopically labelled as described above may be introduced into a biological system, for example by administration to a patient, e.g. a human patient. The cleavage of the polymer may then be monitored by taking samples from the biological system. Where a particular enzyme is unusually expressed, or over-expressed, (e.g. in a disease state) the cleavage of its substrate may be identified by the appearance in a mass spectrum of a unique pattern of peaks. For such a method, the labelled polymer may be given as a single entity or as a mixture of labelled polymers for multi-diagnostic testing. For example, a number of substrates known to be cleaved by the enzyme of interest may be used or a number of substrates each known to be cleaved by a possible enzyme of interest may be used.

The differential isotopic labelling of the polymer in such in vivo, in vitro or ex vivo methods means that the polymer and any potential cleavage products do not need to be extracted and purified and that they can be easily distinguished from any similar or equivalent molecules which are naturally present in the system. Further, the use of isotopic labelling rather than traditional chemical labelling techniques reduces the possibility that the presence of the label will have an influence on the cleavage kinetics.

Polymer Sequencing:

If the structure or sequence of a polymer used in a method of the present invention is unknown (e.g. split-pool synthesis, deletion synthesis, pooled screening strategies) the polymer, or its cleaved fragments, may be subsequently (or simultaneously) sequenced by established methods. This may be used to identify a specific cleavage site and/or to obtain the sequence or structure of those polymers which have been cleaved.

As described above, individual cleaved fragments may be identified based on a mass spectrum. These fragments may then be further cleaved, for example chemically such as by Edman degradation, or by fragmentation using mass spectrometry techniques such as MS/MS. Such methods of cleavage and fragmentation are known in the art. Analysis of the resultant fragments may be used to determine the structure or sequence of the pair of cleaved fragments. For example, if the polymer is a polypeptide, cleavage between individual amino acids will produce fragments of different sizes depending upon the particular amino acid removed. This data can be used to determine the primary structure of the original polypeptide.

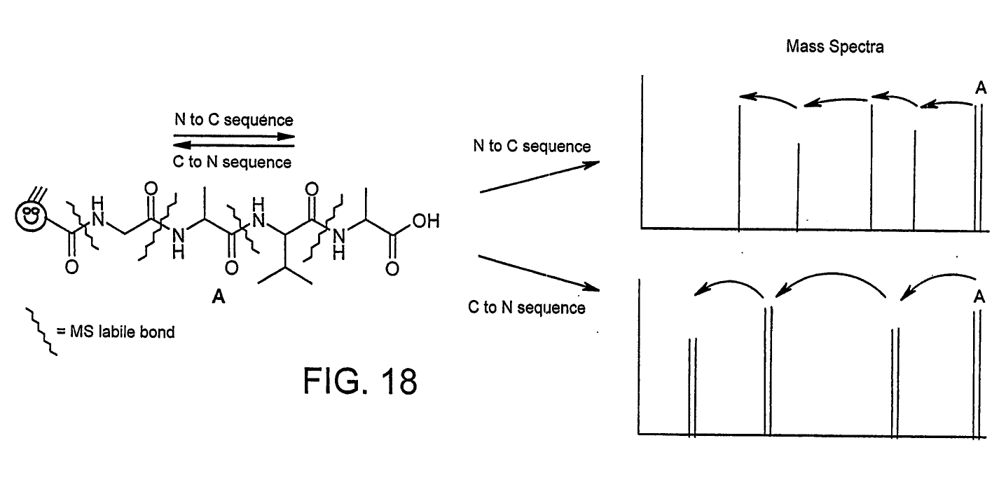

Preferably, the cleaved fragments obtained by the methods of the invention are further analysed using mass spectrometry. Whereas a traditional mass spectrum obtained following fragmentation of a molecule reveals a fingerprint of single masses, the presence of differential isotopic labels offers additional ease of analysis of such fragments. Fragments still comprising an isotopic label may be identified by the presence of pairs of peaks on a mass spectrum. Fragments not comprising the isotopic label may be identified as single peaks on a mass spectrum. If the isotopic label was applied at a terminal of such a polypeptide, sequential cleavage of monomers, e.g. amino acids, from the opposite end of the molecule will result in a series of pairs of peaks on a mass spectrum. The difference in size of consecutive pairs will indicate the size of the monomer (e.g. amino acid) which has been removed, and this data may be used to determine the primary sequence of the original molecule. For example, in a peptide labelled at the N terminal, fragmentation in the C— to N-direction retains the peak split caused by the isotopic label giving rise to doublets, while the reverse fragmentation in the N— to C-direction produces only single mass peaks (see FIG. 18). The use of mass spectrometry to determine the primary structure of a peptide is known in the art (e.g. McLuckey S. A. & Wells J. M., Chem Rev (2001) 101; 571-606; Biemann K., in Biological mass spectrometry, Proceedings of the Second International Symposium on Mass Spectrometry in the Health and Life Sciences, San Francisco, Calif., U.S.A., Elsevier Publishers: Aug. 27-31 1989, p179).

Peak split recognition software (‘cluster analysis’) is available from Micromass© Ltd and has to date been used for analytical construct and bead decoding technologies (McKeown S. C., Watson, S., Carr R., Marshall, Tetrahedron Lett (1999) 40: 2407; Lane S. J., Pipe A. J., Rapid Communications in Mass Spectrometry (2000) 14: 782-793). This software simplifies the data interpretation by filtering out specific ‘peak splits’. This software could also be used to automate the interpretation of the mass spectra obtained using the methods of the present invention.

Kits

The invention further provides kits for use in the methods of the invention.

A kit may be provided for screening a library of polymers for cleavage, for example using a screening method of the invention. A kit may be provided for screening a library of polymers for those polymers which are cleaved by a particular method, for example by a particular enzyme. Such a kit may comprise two or more polymers which have been differentially isotopically labelled as described above. The polymers may be any polymers which are differentially isotopically labelled, for example polymers suitable for use in a method of the invention as are described above.

Suitable polymers for inclusion in a kit of the invention may be supplied in solid form, in liquid form, as a solution or on a solid support. A suitable solid support may be as described above. For example, a kit may comprise two or more resin beads, wherein attached to each bead is one or more molecules of a differentially isotopically labelled polymer as described above. Where polymers are attached to a solid support via a cleavable linker molecule, a kit may further comprise a linker cleaving reagent.

Suitable polymers may further comprise a detectable label on one side of the cleavage site and an inhibitor for that label on the opposite side of the cleavage site. Suitable polymers may comprise a moeity such as biotin which allows separation of the labelled polymer or cleaved fragment from other material.

The present invention also provides kits for the preparation of a polymer suitable for use in a method of the invention. Such a kit may comprise a set of chemical monomers which comprises monomers which have been isotopically labelled as described above. Such a kit will preferably contain monomers which comprise different isotopic labels so that different labels may be added either on each side of the cleavage site of the polymer, or to different polymers to be screened together. Such a kit may further comprise monomers that contain no isotopic label. Preferably the monomers are in a form suitable for polymer synthesis. A kit for the preparation of a polymer suitable for use in a method of the invention may comprise further components, for example means for combining monomers or for adding a monomer to a polymer molecule. For example, a kit may further comprise suitable enzymes or buffers to enable polymerisation to occur.

In a preferred aspect of the invention, a kit for the preparation of a polymer suitable for use in a method of the invention comprises a set of isotopically labelled animo acids. Such a kit may further comprise a set of amino acids which contain no isotopic label. Such a kit may, for example, comprise all twenty proteogenic amino acids in unlabelled form and in one or more isotopically labelled forms. Such a kit may further comprise means for adding an isotopic label to an unlabelled amino acid, for example by modification such as acetylation, hydroxylation, carboxylation or phosphorylation. Such a kit may further comprise isotopically labelled and unlabelled chemical derivitising agents such as Ac2O-d6

In an alternative aspect, a kit for the preparation of a polymer suitable for use in a method of the invention comprises a set of isotopically labelled nucleotides. Such a kit may fierier comprise a set of nucleotides which contain no isotopic label. Such a kit may, for example, comprise a complete set of nucleotides in unlabelled form and in one or more isotopically labelled forms. Such a kit may flirter comprise means for adding an isotopic label to an unlabelled nucleotide, for example by modification such as acetylation, hydroxylation or carboxylation.

### EXAMPLES

General Methods

Mass Spectrometry Analysis:

HPLC was run on a Varian 9010 instrument using a C₈ reverse phase column (Vydac, 22 cm, 0.5 cm φ) and 15 to 90% solvent B gradient (1 ml/min) as the mobile phase. [Solvent A: 1% TFA in water; solvent B; 0.5% TFA in MeCN:water (10:1), 20 min gradient time].

Mass Spectrometry measurements were carried out on a Micromass LCT System orthogonal accelerating Time-of-Flight (oa-TOF MS), in Positive Ion Electrospray mode (ESI,+ve)

| | |
|---|---|
| Ionisation Mode: | Positive Ion Electrospray (ESI, +ve) |
| Acquisition Mode: | TOF MS |
| Scan Range: | 100-1600 amu |
| Scan Rate: | 1.0 sec, 0.10 Interscan delay |
| Flowrate: | 0.400 ml min⁻¹ (no flow split) |
| Runtime: | 30 minutes. |

Analysis of Peptide Purity:

The chromatography was carried out using a Waters Xterra column, (MSC₁₈, 2.1×150 mm, 3.5 μm) 0 to 100% solvent B gradient (0.4 ml/min) as the mobile phase. [Solvent A: 0.1% Formic Acid in water; solvent B; 0.1% Formic Acid in MeCN, 20 min gradient time]. A UV Diode Array (DAD) 190-600 nm system was used for the detection.

| Column: | Waters Xterra, MSC₁₈, 2.1 × 150 mm, 3.5 μm | | |
|---|---|---|---|
| Channel A: | 0.1% (^{v}/ᵥ) Formic acid (aq) | | |
| Channel B: | 100% (^{v}/ᵥ) Acetonitrile + 0.1% (^{v}/ᵥ) Formic acid (aq) | | |
| Gradient: | 0.00 mins | 100% A | 0.00% B |
| | 2.00 | 100 | 0.00 |
| | 18.00 | 0.00 | 100 |
| | 20.00 | 0.00 | 100 |
| | 22.00 | 100 | 0.00 |
| | 30.00 | 100 | 0.00 |

| | |
|---|---|
| Flowrate: | 0.400 ml min⁻¹ |
| Detection: | UV Diode Array (DAD) 190-600 nm |
| Injection Volume: | 5 μl |

Trypsin was obtained from Sigma (reference T1426) and NEP was obtained from BioMep.

### Example 1

### Identification of Cleavage Site

Peptide A-B is an uncleaved molecule which is differentially labelled at both N— and C-termini with stable isotopes. [symbol] =two deuterium (D) or (H) atoms incorporated into Gly, [symbol] =three D or H atoms incorporated into acetate. This uncleaved molecule shows a distinctive mass spectrum of four peaks due to the potential combinations of isotopes within the molecule (FIG. 1).

Following cleavage to fragments A and B, two new pairs of peaks are seen on the mass spectrum (FIG. 1). A pair of peaks with a peak split of 2 AMU (atomic mass units) indicates that the peptide has been cleaved and represents the C-terminal fragment B ( [symbol] ). A pair of peaks with a peak split of 3 AMU indicates that the peptide has been cleaved and represents the N-terminal fragment A ( [symbol] ). Any remaining uncleaved peptide A-B is seen as a remaining quartet of peaks at a higher relative mass. The site of cleavage is readily determined by the mass of the cleaved fragments A and B.

### Example 2

### Enzymatic Cleavage of a Peptide

The peptide Ac-Gly-Pro-Arg-Ala-Ala-Ala-Gly-NH₂ was synthesised. This peptide contains a trypsin cleavage site. This peptide was 50% differentially labelled with three deuterium atoms in place of three hydrogen atoms at the N terminus and with two deuterium atoms in place of two hydrogen atoms at the C terminus.

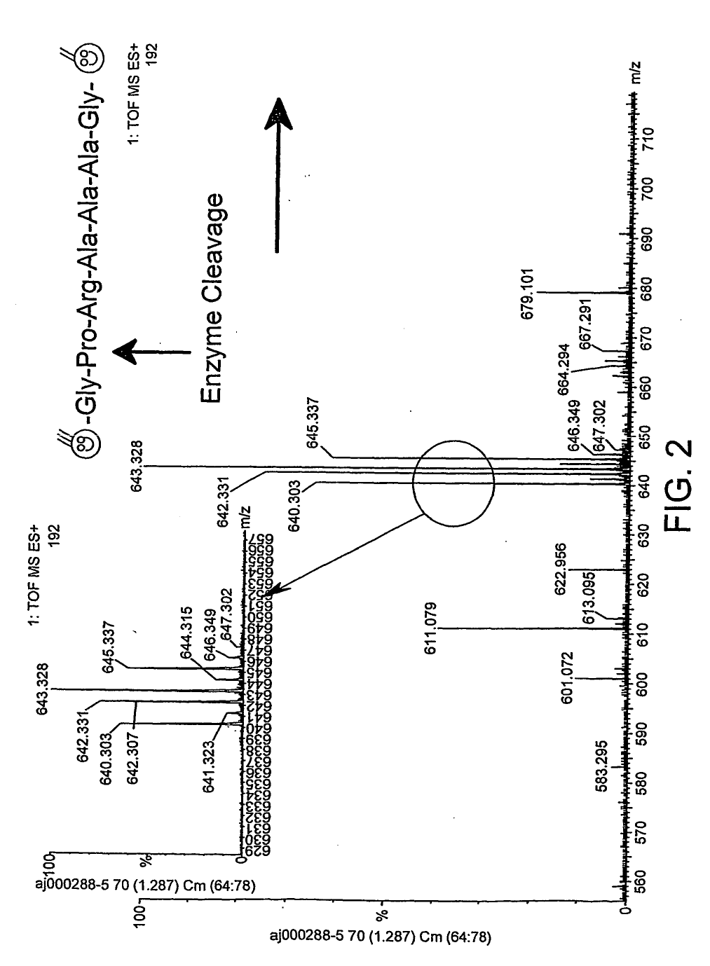

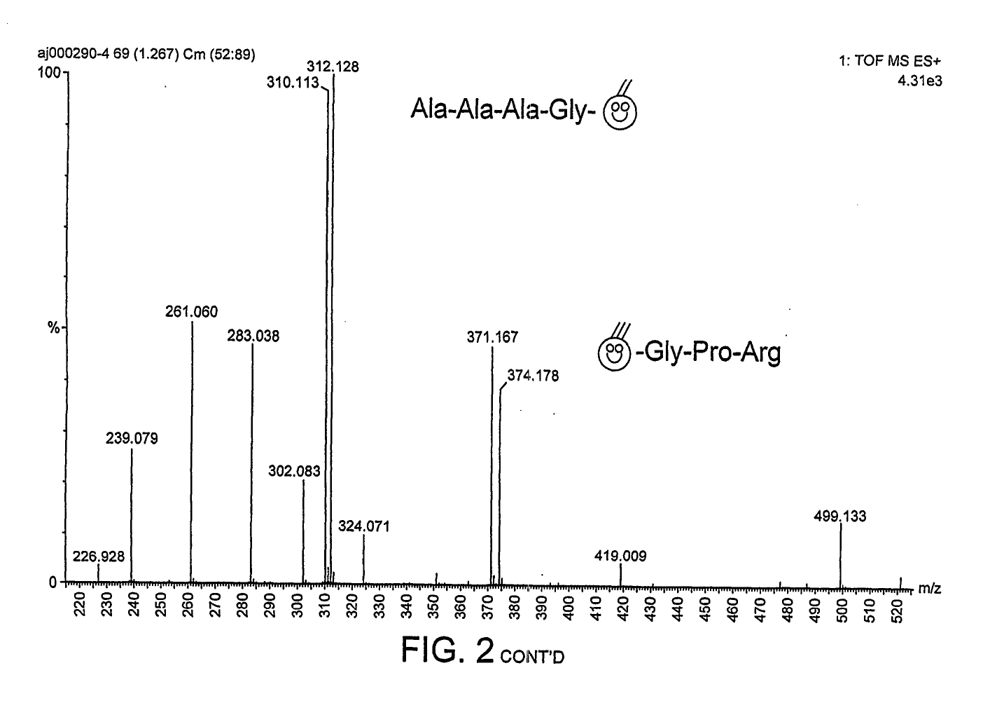

The substrate was incubated with trypsin and mass spectrum analysis performed directly on the ‘incubation’ mixture. FIG. 2 shows the appearance of the two differential peak splits indicating the event of cleavage and secondly, from their individual ‘peak split’ state and masses, identifying its origin (C or N end) and cleavage site.

It has generally been found that since the peptides and deletion peptides synthesised in this manner all have a characteristic ‘quartet’, this may, in some instances, obviate the need to purify some synthesised peptides.

### Example 3

### Synthesis of 6 DiMaS Peptides as Isolated Solids

Six differentially isotopically labelled (DiMaS) peptides were synthesised as described below. The peptides were selected to contain specific enzymatic cleavage sites.
i) Trypsin: 4: AcGly-Pro-Arg-Ala-Ala-Ala-Gly-NH2
ii) α-Secretase: 6: AcSer-Glu-Val-Asn-Leu-Asp-Ala-Glu-Phe-Gly-NH2
iii) β-Secretase: 8: AcGly-Gly-Val-Val-Ile-Ala-Thr-Val-Ile-Gly-NH2
iv) γ secretase: 10: AcHis-His-Gln-Lys-Leu-Val-Phe-Phe-Ala-Gly-NH2
v) Caspase 3: 12: AcGly-Asp-Glu-Val-Asp-Gly-NH2
vi) Caspase 1: 14: AcTyr-Val-Ala-Asp-Ala-Pro-Val-Gly-NH2

Commercially available Rink amide resin (3 g, 1.86 10⁻³ mol) was treated with 20% Piperidine in DMF and shaken for 1 h, the resin was then drained and washed with DMF (3×5 ml), DCM (3×5 ml), DMF (3×5 ml), DMF (3×5 ml), Et₂O (3×5 ml), DCM (3×5 ml) and finally Et₂O (3×5 ml) and dried in vacuo. PyBop (4.84 g, 9.3 10⁻³ mol), DIPEA (3.2 ml, 1.86 10⁻² mol), and Fmoc-gly-OH (D₂) (2.76 g, 9.3 10⁻³ mol) were mixed in NMP (15 ml) and added to the dried resin and shaken for 1 h and then drained and washed as above. The resin was then treated with 20% Piperidine in DMF and shaken for 1 h, the resin was then drained and washed as above to give resin 1.

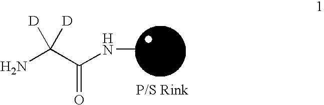

Commercially available Rink amide resin (3 g, 1.86 10⁻³ mol) was treated with 20% piperidine in DMF and shaken for 1 h, the resin was then drained and washed with DMF (3×5 ml), DCM (3×5 ml). DMF (3×5 ml), DMF (3×5 ml), Et₂O (3×5 ml), DCM (3×5 ml) and finally Et₂O (3×5 ml) and dried in vacuo. PyBop (4.84 g, 9.3 10⁻³ mol), DIPEA (3.2 ml, 1.86 E-2 mol), and Fmoc-gly-OH (2.76 g, 9.3 10⁻³ mol) were mixed in NMP (15 ml) and added to the dried resin and shaken for 1 h and then drained and washed as above. The resin was then treated with 20% piperidine in DMF and shaken for 1 h, the resin was then drained and washed as above to give resin 2.

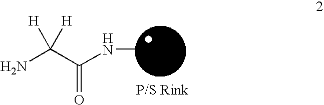

Resins 1 (130 mg) and 2 (130 mg) were mixed and the sequence Gly-Pro-Arg-Ala-Ala-Ala was synthesised using an ABI433 No.1 Synthesiser using HBTU/Fmoc protocol and standard side chain protection group strategy. A portion of the final amino resin (50 mg) was then treated with acetic anhydride (containing 50% D₆-Ac₂O) (0.2 ml) in 1:1 DCM/DMF (1 ml) and shaken for 2 h. The resin was then drained and washed with DMF (3×5 ml), DCM (3×5 ml), DMF (3×5 ml), DMF (3×5 ml), Et₂O (3×5 ml), DCM (3×5 ml) and finally Et₂O (3×5 ml) and dried in vacuo to give 3.

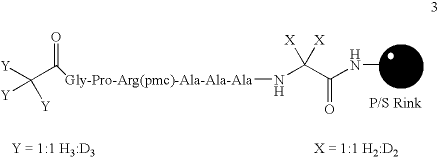

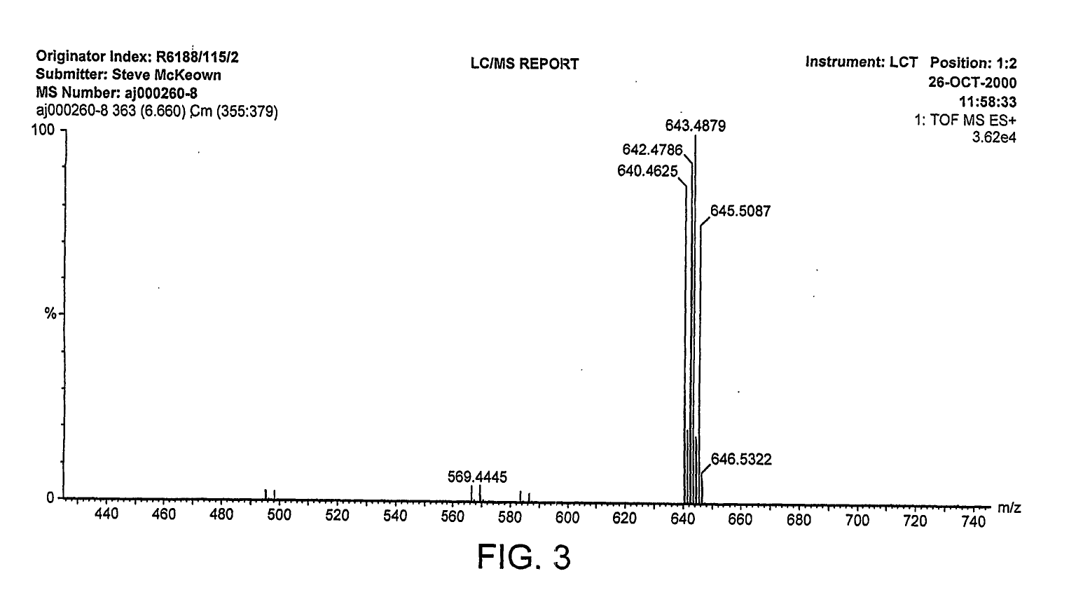

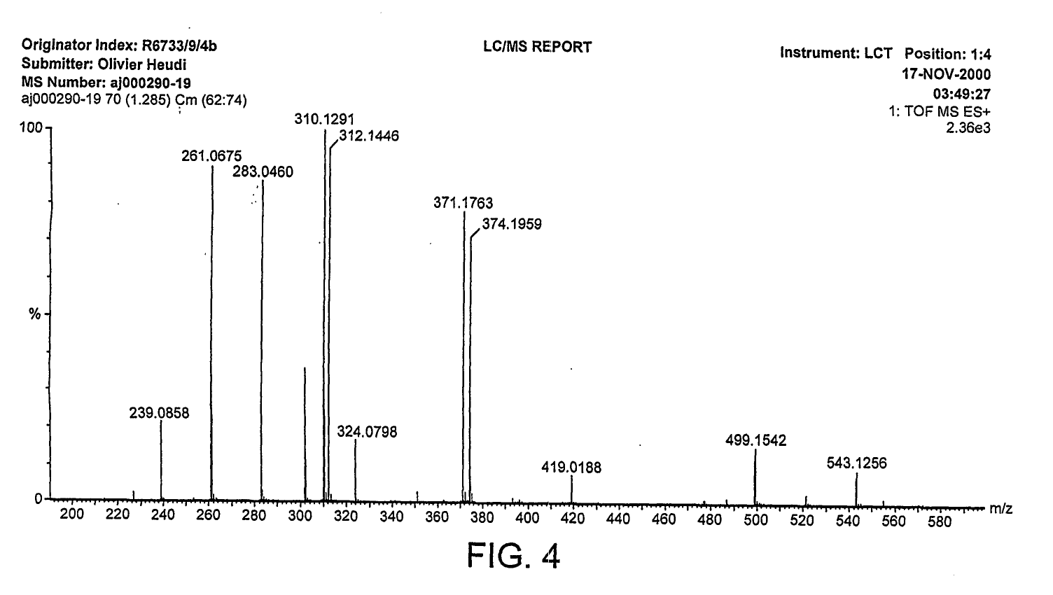

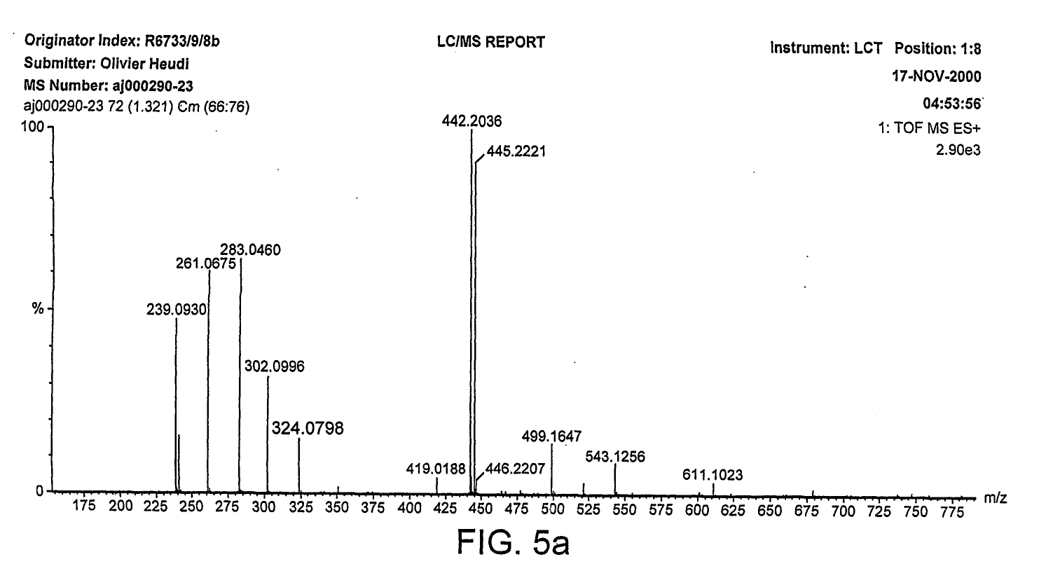

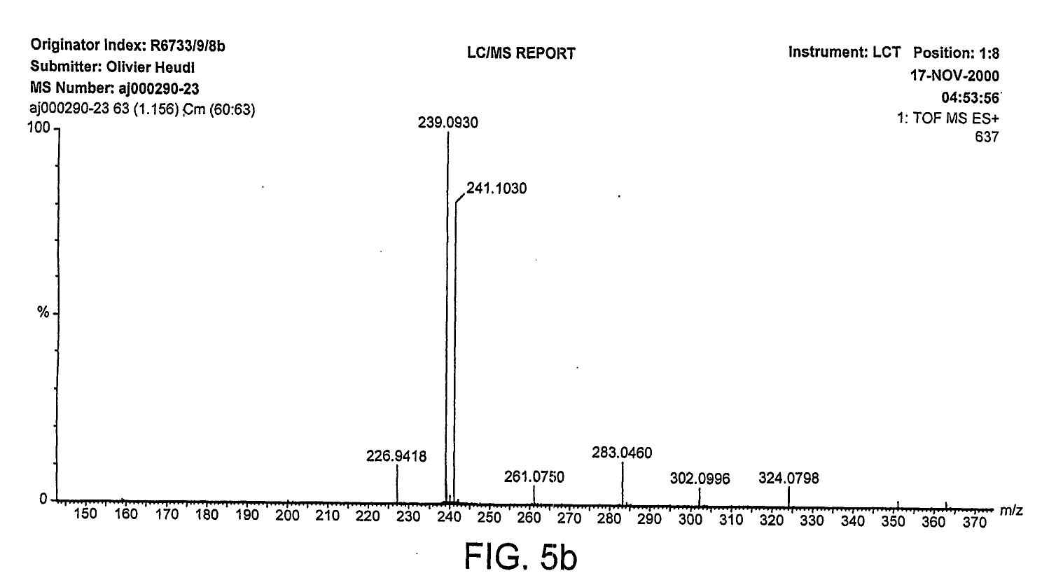

Resin 3 (50 mg) was cleaved by adding 3:0.2 TFA/TIPS (1 ml) at 0 C for 10 min then shaking at rt. for 2 h. The resin was drained into a flask, washed with DCM (2×0.5 ml) and the volatiles evaporated in vacuo. The residue was redissolved in DCM (1 ml) and again evaporated to remove the last traces of TFA (this procedure was repeated five times). The residue was then trituated with Et₂O (5×1 ml). The residue was then dissolved in H₂O (1 ml) and freeze-dried to give a 4 as a white solid. 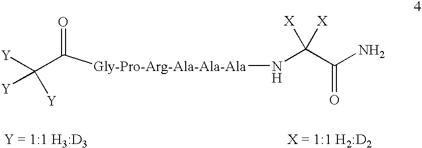
HPLC/MS analysis of 4 is shown in FIG. 3 (aj000260-8)

Resins 1 (130 mg) and 2 (130 mg) were mixed and the sequence Ser-Glu-Val-Asn-Leu-Asp-Ala-Glu-Phe was synthesised using an ABI433 No.1 synthesiser using HBTU/Fmoc protocol and standard side chain protection group strategy. A portion of the final amino resin (50 mg) was then treated with acetic anhydride (containing 50% D₆-Ac₂O) (0.2 ml) in 1:1 DCM/DMF (1 ml) and shaken for 2 h. The resin was then drained and washed with DMF (3×5 ml), DCM (3×5 ml), DMF (3×5 ml), DMF (3×5 ml), Et₂O (3×5 ml), DCM (3×5 ml) and finally Et₂O (3×5 ml) and dried in vacuo to give 5. Resin 5 (50 mg) was cleaved by adding 3:0.2 TFA/TIPS (1 ml) at 0 C for 10 ins then shaking at rt. for 2 h. The resin was drained into a flask, washed with DCM (2×0.5 ml) and the volatiles evaporated in vacuo. The residue was redissolved in DCM (1 ml) and again evaporated to remove the last traces of TFA (this procedure was repeated five times). The residue was then trituated with Et₂O (5×1 ml). The residue was then dissolved in H₂O/dioxane and freeze-dried to give a 6 as a white solid.

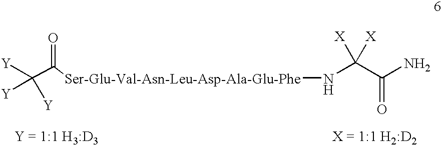

Resins 1 (130 mg) and 2 (130 mg) were mixed and the sequence Gly-Gly-Val-Val Ile-Ala-Thr-Val-Ile was synthesised using an ABI433 No.1 synthesiser using HBTU/Fmoc protocol and standard side chain protection group strategy. A portion of the final amino resin (50 mg) was then treated with acetic anhydride (containing 50% D₆-Ac₂O) (0.2 ml) in 1:1 DCM/DMF (1 ml) and shaken for 2 h. The resin was then drained and washed with DMF (3×5 ml), DCM (3×5 ml), DMF (3×5 ml), DMF (3×5 ml), Et₂O (3×5 ml), DCM (3×5 ml) and finally Et₂O (3×5 ml) and dried in vacuo to give 7. Resin 7 (50 mg) was cleaved by adding 3:0.2 TFA/TIPS (1 ml) at 0 C for 10 ins then shaking at rt. for 2 h. The resin was drained into a flask, washed with DCM (2×0.5 ml) and the volatiles evaporated in vacuo. The residue was redissolved in DCM (1 ml) and again evaporated to remove the last traces of TFA (this procedure was repeated five times). The residue was then trituated with Et₂O (5×1 ml). The residue was then dissolved in H₂O/dioxane (2 ml) and freeze-dried to give 8 as a white solid.

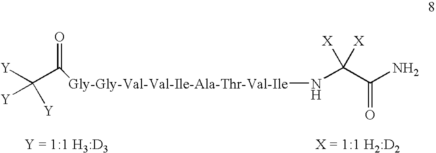

Resins 1 (130 mg) and 2 (130 mg) were mixed and the sequence His-His-Gln-Lys-Leu-Val-Phe-Phe-Ala was synthesised using an ABI433 No.1 synthesiser using HBTU/Fmoc protocol and standard side chain protection group strategy. A portion of the final amino resin (50 mg) was then treated with acetic anhydride (containing 50% D₆Ac₂O) (0.2 ml) in 1:1 DCM/DMF (1 ml) and shaken for 2 h. The resin was then drained and washed with DMF (3×5 ml), DCM (3×5 ml), DMF (3×5 ml), DMF (3×5 ml), Et₂O (3×5 ml), DCM (3×5 ml) and finally Et₂O (3×5 ml) and dried in vacuo to give 9. Resin 9 (50 mg) was cleaved by adding 3:0.2 TFA/TIPS (Ill) at 0 C for 10 mins then shaking at room temperature for 2 h. The resin was drained into a flask, washed with DCM (2×0.5 ml) and the volatiles evaporated in vacuo. The residue was redissolved in DCM (1 ml) and again evaporated to remove the last traces of TFA (this procedure was repeated five times). The residue was then trituated with Et₂O (5×1 ml). The residue was then dissolved in H₂O/dioxane (2 ml) and freeze-dried to give a 10 as a white solid.

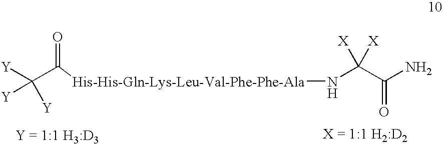

Resins 1 (130 mg) and 2 (130 mg) were mixed and the sequence Gly-Asp-Glu-Val-Asp was synthesised using an ABI433 No.1 Synthesiser using HBTU/Fmoc protocol and standard side chain protection group strategy. A portion of the final amino resin (50 mg) was then treated with acetic anhydride (containing 50% D₆-Ac₂O) (0.2 ml) in 1:1 DCM/DMF (1 ml) and shaken for 2 h. The resin was then drained and washed with DMF (3×5 ml), DCM (3×5 ml), DMF (3×5 ml), DMF (3×5 ml), Et₂O (3×5 ml), DCM (3×5 ml) and finally Et₂O (3×5 ml) and dried in vacuo to give resin 11. Resin 11 (50 mg) was cleaved by adding 3:0.2 TFA/TIPS (1 ml) at 0 C for 10 mins then shaking at room temperature for 2 h. The resin was drained into a flask, washed with DCM (2×0.5 ml) and the volatiles evaporated in vacuo. The residue was redissolved in DCM (1 ml) and again evaporated to remove the last traces of TFA (this procedure was repeated five times). The residue was then trituated with Et₂O (5×1 ml). The residue was then dissolved in H₂O (1 ml) and freeze-dried to give a 12 as a white solid.

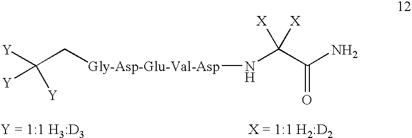

Resins 1 (130 mg) and 2 (130 mg) were mixed and the sequence Tyr-Val-Ala-Asp-Ala-Pro-Val was synthesised using an ABI433 No.1 Synthesiser using HBTU/Fmoc protocol and standard side chain protection group strategy. A portion of the final amino resin (50 mg) was then treated with acetic anhydride (containing 50% D₆-Ac₂O) (0.2 ml) in 1:1 DCM/DMF (1 ml) and shaken for 2 h. The resin was then drained and washed with DMF (3×5 ml), DCM (3×5 ml), DMF (3×5 ml), DMF (3×5 ml), Et₂O (3×5 ml), DCM (3×5 ml) and finally Et₂O (3×5 ml) and dried in vacuo to give resin 13. Resin 13 (50 mg) was cleaved by adding 3:0.2 TFA/TIPS (1 ml) at 0 C for 10 mins then shaking at room temperature for 2 h. The resin was drained into a flask, washed with DCM (2×0.5 ml) and the volatiles evaporated in vacuo. The residue was redissolved in DCM (1 ml) and again evaporated to remove the last traces of TFA (this procedure was repeated five times). The residue was then trituated with Et₂O (5×1 ml). The residue was then dissolved in H₂O and freeze-dried to give a 14 as a white solid.

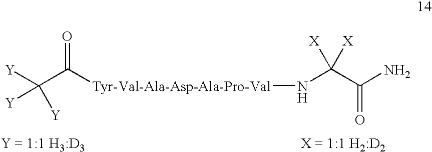

### Example 4

### Incubation of Peptides with NEP and Trypsin

Incubation with Trypsin.

The peptide (10 μl of 1 mM in Acetonitrile) was treated with a solution of buffer (50 mM HEPES, 150 mM NaCl and 1 μM Zn²⁺, 35 μl), and finally Trypsin was added (5 μl, 2.5 μM). The reaction was allowed to take place at room temperature for 9 h and an aliquot of solution was removed for LC-MS analysis.

Incubation with NEP.

The peptide (10 μl of 1 mM in Acetonitrile) was treated with a solution of buffer (50 mM HEPES, 150 mM NaCl and 1 μM Zn²⁺, 35 μl), and finally neutral endopeptidase (NEP) was added (5 μl, 2.5 μM). The reaction was allowed to take place at room temperature for 9 h and an aliquot of solution was removed for LC-MS analysis.

Peptides 4 and 10 were incubated with trypsin as described above and then analysed by HPLC/MS to give masses according to Table 1.

Peptides 4, 6 and 10 were incubated with NEP as described above and then analysed by HPLC/MS to give masses according to Table 1.

TABLE 1

| | | Relevant Masses | |
|---|---|---|---|
| Pep- | Incubation | (M + H or M + Na) (ignoring | |
| tide | Enzyme | natural ¹³C abundance) | Figures |
| 4 | No Incubation | 640, 642, 643, 645 | 3 (aj000260-8) |
| 6 | No Incubation | 1121, 1123, 1124, 1126 | |
| 8 | No Incubation | 926, 928, 929, 931 | |
| 10 | No Incubation | 1225, 1227, 1228, 1230 | |
| 12 | No Incubation | 632, 634, 635, 637 | |
| 14 | No Incubation | 832, 834, 835, 837 | |
| 4 | Trypsin | 371, 374: AcGlyProArgOH | 4 |
| | | 310, 312: | (aj000290-19) |
| | | H₂NalaAlaAlaGlyNH₂ | |
| | | (Na+) | |
| 10 | Trypsin | 591, 594 AcHisHisGlnLysOH | |
| | | 652, 654: | |
| | | H₂NleuValPhePheAlaGlyNH₂ | |
| 4 | NEP | 442, 445: | 5a |
| | | AcGlyProArgAlaOH | (aj000290-23) |
| | | 239, 241: H₂NAlaAlaGlyNH₂ | 5b |
| | | (Na+) | (aj000290-23a) |
| 6 | NEP | 650, 652: | |
| | | H₂NleuAspAlaGluPheGlyNH₂ | |
| 10 | NEP | 704, 707: | |
| | | AcHisHisGlnLysLeuOH | |
| | | 315, 317: NH₂PheAlaGlyNH₂ | |

### Example 5

### Fluorescent On-Bead Peptide Screening

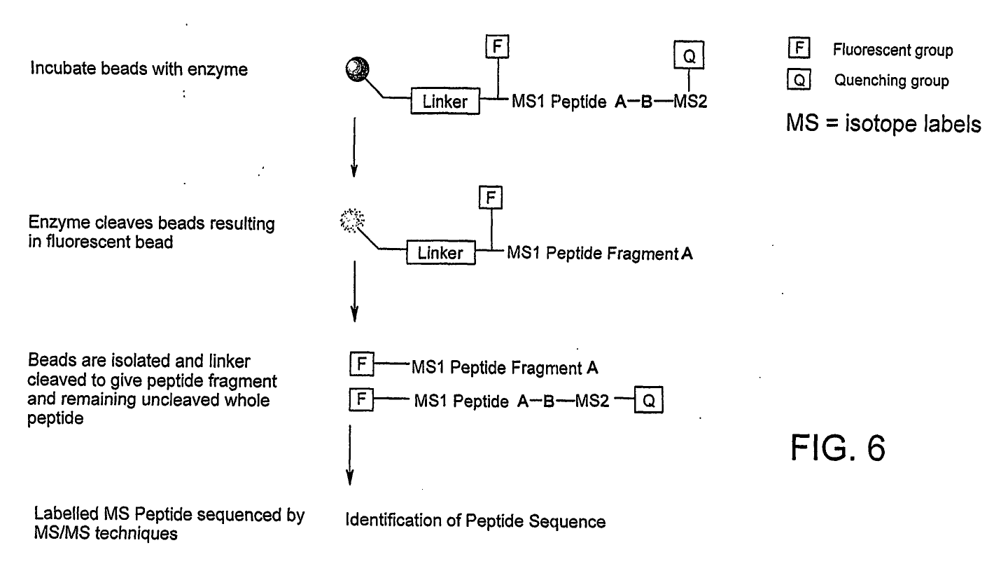

A library of peptides may be synthesised on resin beads that incorporate fluorescent and quenching groups. This library may then be used to screen for peptides which are cleaved by a particular enzyme (FIG. 6). The library may be designed such that after peptide cleavage with an enzyme, the resulting bead emits a fluorescent signal on irradiation. This fluorescence signals the event of cleavage. The recognisable bead can be selected and further analysed. The ratio of fluorophore (F) and quenching agent (Q) can be optimised to give zero emission before incubation and a positive fluorescence on hydrolysis of the peptide (R_{foz} see FIG. 7). This is important since too much initial quenching agent could still prevent emission even when significant cleavage has taken place.

The cleaved (MS isotopically labelled) peptide is then released via the linker along with any remaining, uncleaved peptide and subjected to MS fragmentation, confirming cleavage, identifying their sequences and identifying the site of hydrolysis (FIG. 6).

This approach is amenable to screening all the peptides in one vessel. For example, 1 g of resin beads can be incubated together with the enzyme, subsequently washed of assay impurities, irradiated and the fluorescent beads removed by hand or picked via robotic automation. The ‘active’ substrates are then identified as described above. 1 g of resin beads may contain approximately V2 million beads, making this approach attractive for diverse screening methods.

### Example 6

### Synthesis of Polymer Supported DiMaS Peptide

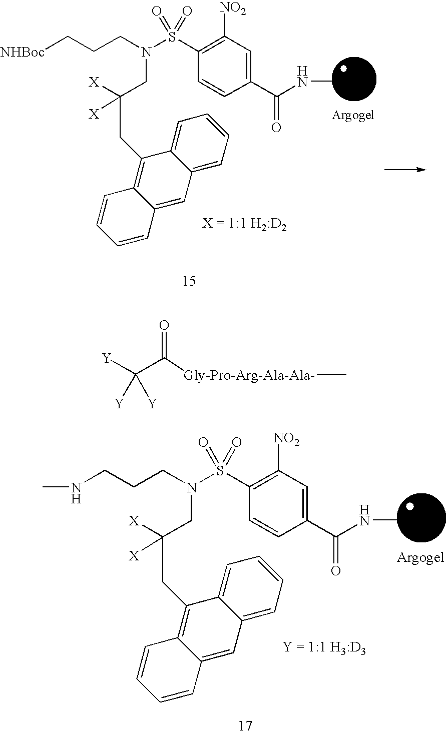

Resin 15 was prepared in a similar method to that described in the literature: (G. M. Williams et al. Angew. Chem., Int. Ed. Eng. 2000, 39 (18), 3293. Congreve et al. Org lett 2001, 3(4), 507)

Resin 15 (50 mg, 0.43 mmol/g) was treated with 95% TFA (aq) for 2 h. The TFA was drained and the resin washed with DCM, DMF, DCM, 10% DIPEA in DMF, DCM. Each of the amino acids were assembled as follows: A preformed activated ester of the amino acid was formed by mixing the Fmoc amino acid (0.43 mmol) with HATU (0.43 mmol) and DIPEA (0.86 mmol) in DMF (2 ml) for 10 min, this was then added to the resin and shaken for 4 h. The resin was then drained and washed with DMF, DCM, DMF and DCM (all ×5). 20% Piperidine in DMF (5 ml) was then added to the resin and the resin shaken for 1 h. The resin was then drained and washed with DMF,DCM,DMF,DCM (all 5×5 ml).

The sequence Gly-Pro-Arg-Ala-Ala was coupled on to the resin according to the above procedure. The resin was then treated with 10% Ac₂O/Ac(D₃)2O (1:1) in DCM and shaken for 2 h. The resin was then washed with DCM, DMF, DCM (all ×5) to give resin 16.

To remove all the pmc protecting group, the resin was shaken with 95% TFA (aq) for 2 h. The resin was then drained and washed with DCM, 10% DIPEA in DMF, DCM to give resin 17.

A solution of Mercaptoethanol (0.1 ml), DBU (0.33 ml) in Acetonitrile (1.5 ml) was prepared 30 mins before use. The resin (typically 50 beads) is treated with this solution (0.2 ml) and left to stand for 2 h. The supernatant liquid is then removed and analysed via HPLC/MS.

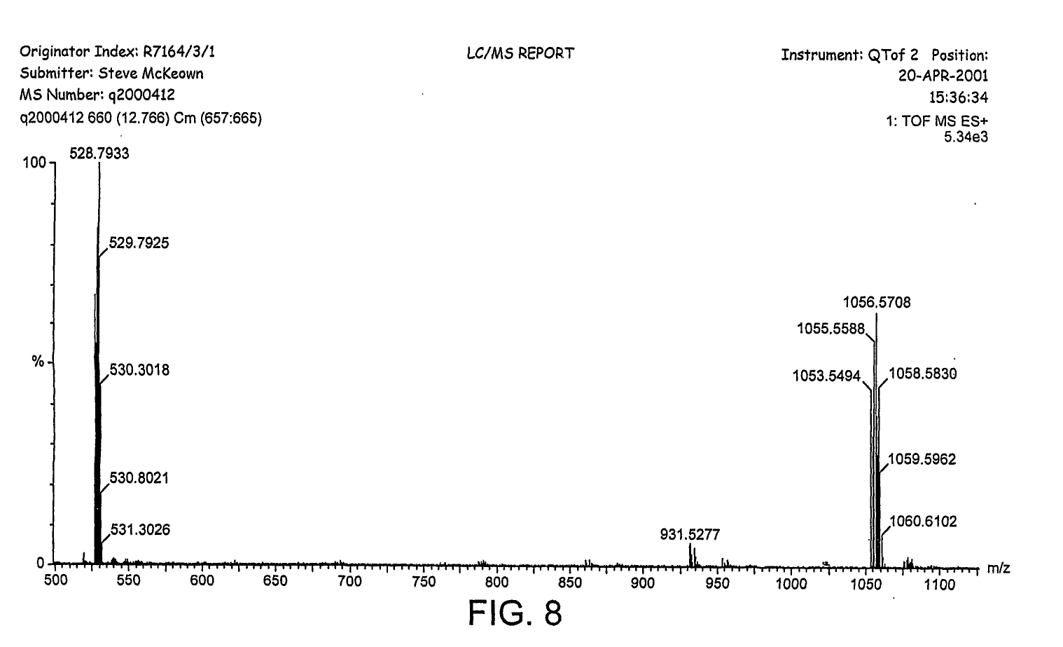

The analysis for resin 16 is shown in FIG. 8.

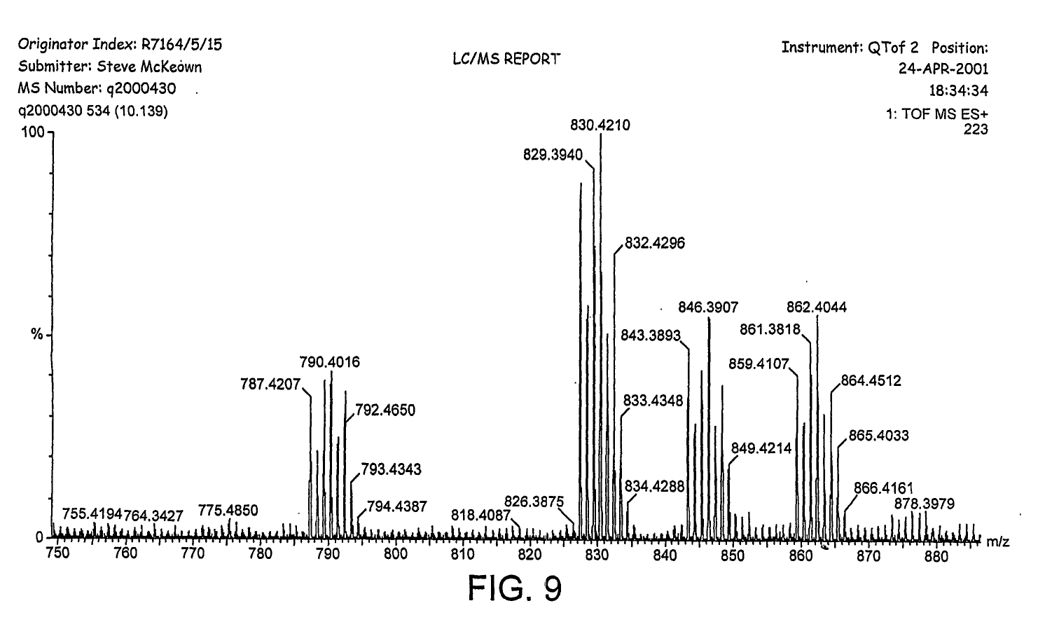

The analysis for resin 17 is shown in FIG. 9.

### Example 7

### On-Bead Incubation of Supported DiMaS Peptide

Incubation of Resin with Trypsin.

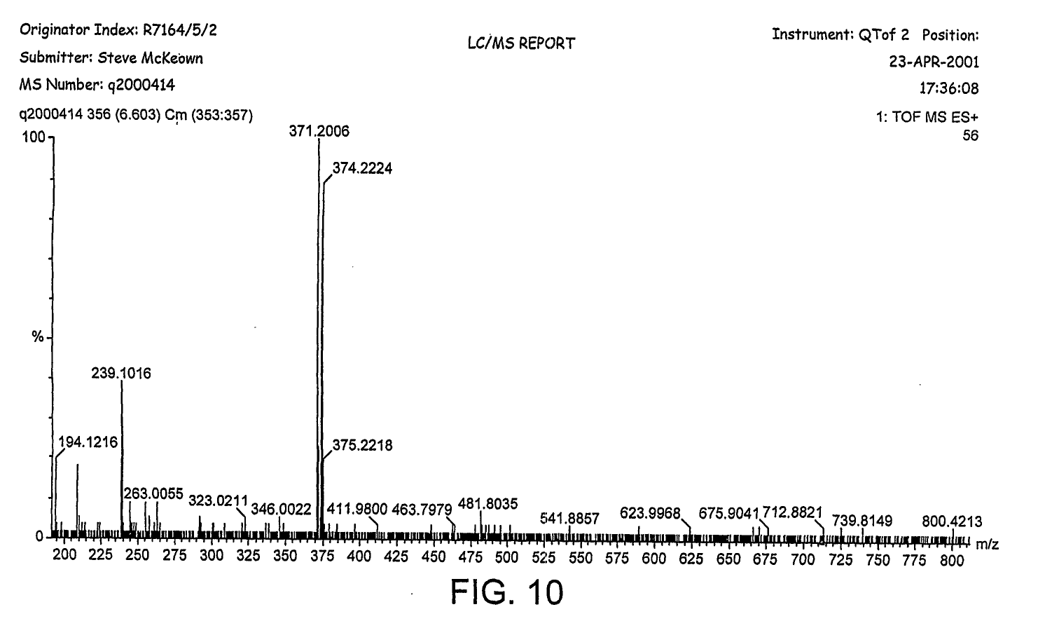

The Resin 15 (ca 20 beads) were treated with a solution of buffer (50 mM HEPES, 150 mM NaCl and 1 C Zn²⁺, 35 μl), and finally Trypsin was added (5 μl, 2.5 μM). The reaction was allowed to take place at room temperature for 2 h and an aliquot of solution was removed for LC-MS analysis. The analysis is shown in FIG. 10.

### Example 8

### Preparation of DiMaS Tagged Peptide Libraries

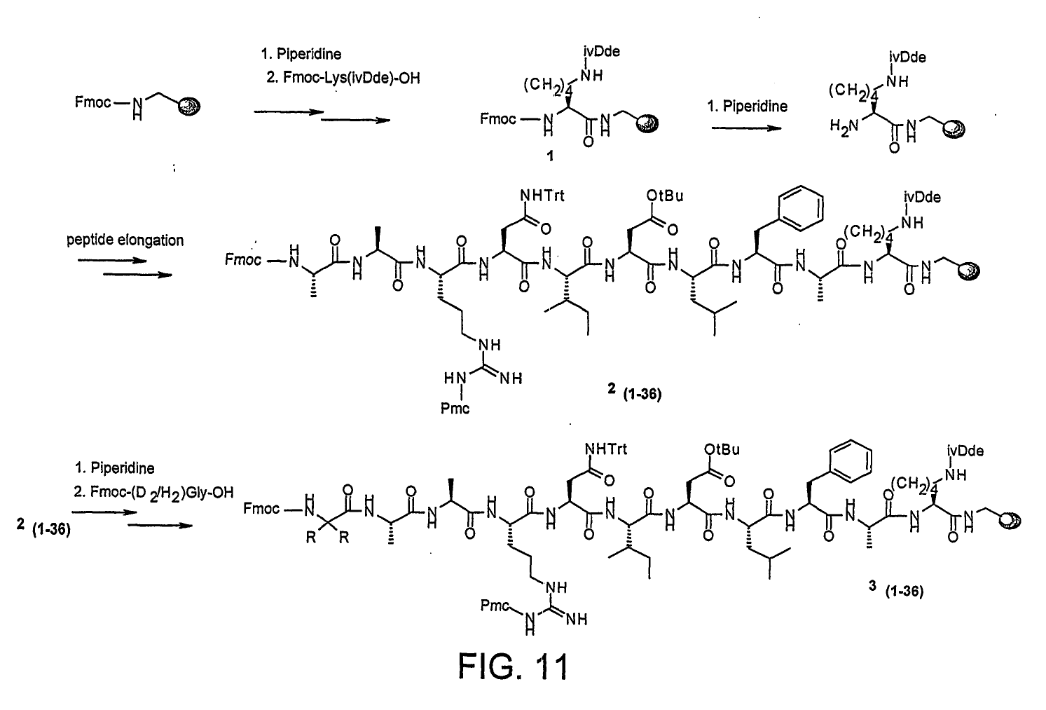

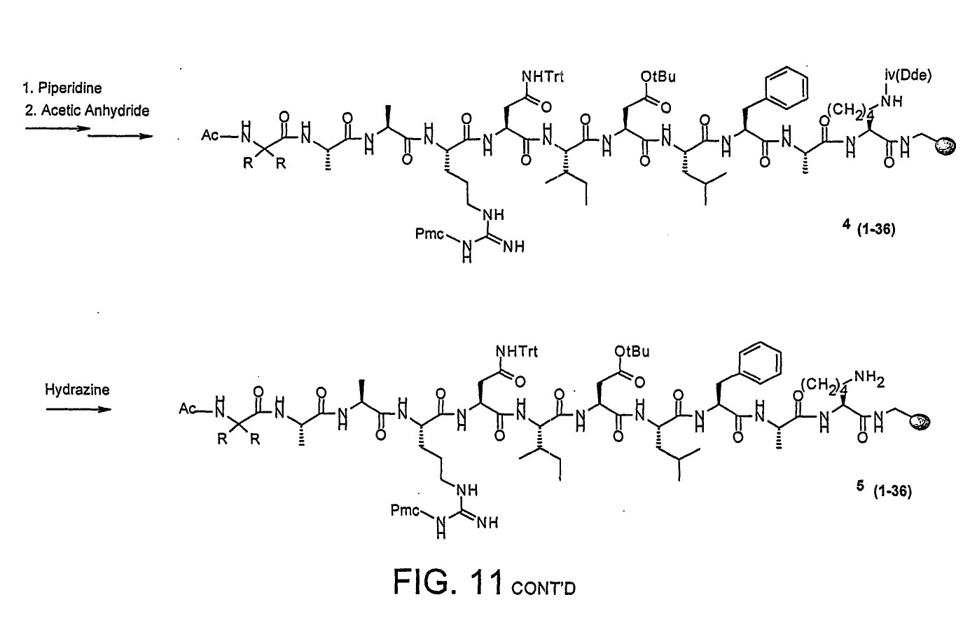

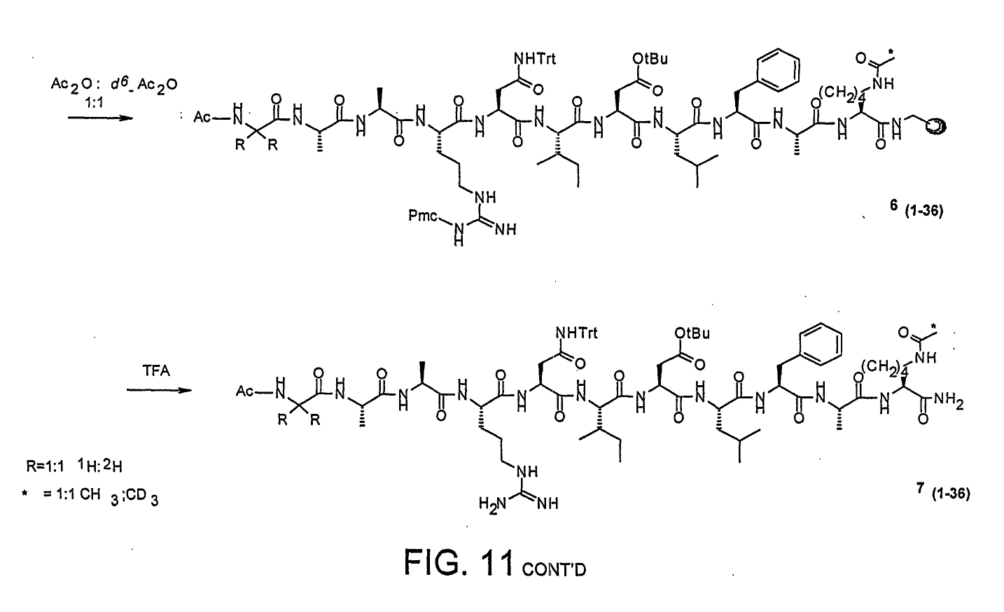

A series of compounds were synthesised using the general strategy outlined in FIG. 11.

Compound 1:

The Fmoc protected Rink Amide resin (3.900 g, 2.34 mmol) was treated with a 20% Piperidine solution in DMF (10 ml) for (2×10 min) and then washed with DMF (6×10 ml). The freshly prepared deprotected resin was then treated with Fmoc-Lys(ivDde)-OH (3.350 g, 5.85 mmol), HBTU (2.210 g, 5.85 mmol), and HOBt (0.790 g, 5.85 mmol) in DMF (10 ml). Finally, Hünig's base (1.510 g, 11.70 mmol) was added and the resin stirred at room temperature for 16 h. The resin was then washed successively with DMF (6×10 ml), dichloromethane (4×10 ml) and ether (2×10 ml), then dried in vacuo to give compound 1 (4.600 g, 2.34 mmol) as a yellow solid. Kaiser test (Kaiser, E.; Colescott, R. L.; Bossinger, C. D.; Cook, P. I. Anal. Biochem., 1970, 34, 595) was negative, Fmoc number 100%. 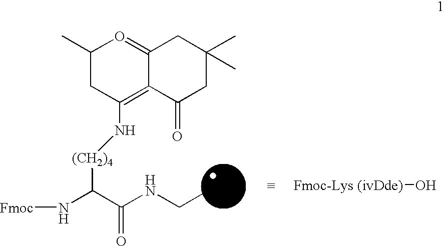
Compounds 2₍₁₋₃₆₎:

The linear peptide was assembled on an automated ACT 357 MPS synthesiser using the standard protocol described by Sheppard¹. The 36 resin aliquots were loaded manually into a standard (6×6) reaction block. The Fmoc protected Rink amide resin (0.12 g, 0.06 mmol) was treated with a 20% Piperidine solution in DMF (2×2 ml, 2×10 min) and then washed with NMP (10×2 ml). The deprotected resin was then treated with Fmoc-Ala-OH (0.056 g, 0.18 mmol) in NMP (700 μl) then HBTU (0.068 g, 0.18 mmol) and HOBt (0.024 g, 0.18 mmol) in NMP (180 μl). Finally Hünig's base (62 μl, 0.36 mmol) in DMF (360 μl) was added. The reaction block was agitated intermittently for 2 h after which the resin was drained under vacuum. A second coupling was performed using the same quantity of reagents as in the first coupling and finally the resin was washed with NMP (6×2 ml). The above procedure was repeated for the incorporation of Fmoc-Phe-OH, Fmoc-Leu-OH, Fmoc-Asp(OtBu)-OH, Fmoc-Ile-OH, Fmoc-Asn(Trt)-OH, Fmoc-Arg(Pmc)-OH, Fmoc-Ala-OH and Fmoc-Ala-OH respectively. The resin was finally washed with NMP (6×2 ml) then manually washed with DCM (2×2 ml) and ether (1×2 ml) then dried in vacuo to give the protected peptide resin 2₍₁₎ as a pale yellow solid (0.30 g, 100%). Kaiser Test was negative.

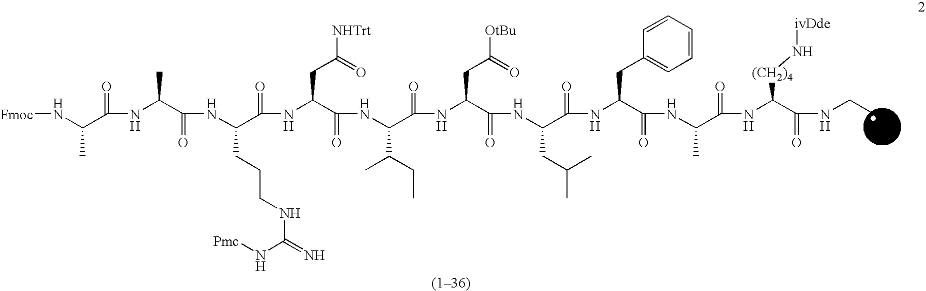

Compounds 2₍₂₋₃₆₎ were prepared in an analogous manner to compound 2₍₁₎ in the remainder of the wells in the ACT reaction block. The structures of compounds 2₍₁₋₃₆₎ are given in Table 2.

TABLE 2
Structure of compounds 2_{(1-36).}

| | | | | | | | | | | |
|---|---|---|---|---|---|---|---|---|---|---|
| 2(1) | Fmoc-Ala | Ala | Arg | Asn | Ile | Asp | Leu | Phe | Ala | Lys(ivDde)- |
| | | | (Pmc) | (Trt) | | (tBu) | | | | Rink Resin |
| 2(2) | Fmoc-Ala | Gly | Arg | Ala | Asn | Ile | Asp | Phe | Ala | Lys(ivDde)- |
| | | | (Pmc) | | (Trt) | | (tBu) | | | Rink Resin |
| 2(3) | Fmoc-Ala | Leu | Arg | Phe | Ala | Asn | Ile | Phe | Ala | Lys(ivDde)- |
| | | | (Pmc) | | | (Trt) | | | | Rink Resin |
| 2(4) | Fmoc-Ala | Phe | Arg | Phe | Ala | Ala | Asn | Ile | Ala | Lys(ivDde)- |
| | | | (Pmc) | | | | (Trt) | | | Rink Resin |
| 2(5) | Fmoc-Ala | Lys | Arg | Ala | Gly | Ala | Ala | Phe | Ala | Lys(ivDde)- |
| | | (Boc) | (Pmc) | | | | | | | Rink Resin |
| 2(6) | Fmoc-Ala | Gln | Arg | Lys | Val | Gly | Ala | Phe | Ala | Lys(ivDde)- |
| | | (Trt) | (Pmc) | (Boc) | | | | | | Rink Resin |
| 2(7) | Fmoc-Ala | Pro | Arg | Ser | Asp | Val | Gly | Phe | Ala | Lys(ivDde)- |
| | | | (Pmc) | (OtBu) | (tBu) | | | | | Rink Resin |
| 2(8) | Fmoc-Ala | Pro | Arg | Phe | Ser | Asp | Val | Leu | Ala | Lys(ivDde)- |
| | | | (Pmc) | | | (tBu) | | | | Rink Resin |
| 2(9) | Fmoc-Ala | Pro | Lys | Ile | Gly | Ser | Asp | Phe | Ala | Lys(ivDde)- |
| | | | (Boc) | | | (OtBu) | (tBu) | | | Rink Resin |
| 2(10) | Fmoc-Ala | Phe | Lys | Ile | Ile | Gly | Ser | Phe | Ala | Lys(ivDde)- |
| | | | (Boc) | | | | (OtBu) | | | Rink Resin |
| 2(11) | Fmoc-Ala | Arg(Pmc) | Lys | Phe | Ile | Ile | Ile | Phe | Ala | Lys(ivDde)- |
| | | | (Boc) | | | | | | | Rink Resin |
| 2(12) | Fmoc-Ala | Asp(tBu) | Lys | Ile | Phe | Ser | Gly | Phe | Ala | Lys(ivDde)- |
| | | | (Boc) | | | (OtBu) | | | | Rink Resin |
| 2(13) | Fmoc-Ala | Phe | Leu | Ala | Arg | Asn | Ile | Asp | Leu | Lys(ivDde)- |
| | | | | | (Pmc) | (Trt) | | (tBu) | | Rink Resin |
| 2(14) | Fmoc-Ala | Phe | Glu | Gly | Arg | Ala | Asn | Ile | Asp | Lys(ivDde)- |
| | | | | | (Pmc) | | (Trt) | | (tBu) | Rink Resin |
| 2(15) | Fmoc-Ala | Ser | Val | Leu | Arg | Phe | Ala | Asn | Ile | Lys(ivDde)- |
| | | (OtBu) | | | (Pmc) | | | (Trt) | | Rink Resin |
| 2(16) | Fmoc-Ala | Ala | Pro | Phe | Arg | Phe | Ala | Ala | Asn | Lys(ivDde)- |
| | | | | | (Pmc) | | | | (Trt) | Rink Resin |
| 2(17) | Fmoc-Ala | Ala | Phe | Lys | Arg | Ala | Gly | Ala | Ala | Lys(ivDde)- |
| | | | | (Boc) | (Pmc) | | | | | Rink Resin |
| 2(18) | Fmoc-Ala | Phe | Lys | Gln | Arg | Lys | Val | Gly | Ala | Lys(ivDde)- |
| | | | (Boc) | | (Pmc) | | | | | Rink Resin |
| 2(19) | Fmoc-Ala | Asp | Ala | Pro | Arg | Ser | Asp | Val | Gly | Lys(ivDde)- |
| | | (tBu) | | | (Pmc) | (OtBu) | (tBu) | | | Rink Resin |
| 2(20) | Fmoc-Ala | Phe | Gly | Pro | Arg | Phe | Ser | Asp | Val | Lys(ivDde)- |
| | | | | | (Pmc) | | (OtBu) | | | Rink Resin |
| 2(21) | Fmoc-Ala | Phe | Ala | Pro | Lys | Ile | Gly | Ser | Asp | Lys(ivDde)- |
| | | | | | (Boc) | | | (OtBu) | (tBu) | Rink Resin |
| 2(22) | Fmoc-Ala | Ala | Pro | Phe | Lys | Ile | Ile | Gly | Ser | Lys(ivDde)- |
| | | | | | (Boc) | | | | (OtBu) | Rink Resin |
| 2(23) | Fmoc-Ala | Gly | Val | Arg | Lys | Phe | Ile | Ile | Gly | Lys(ivDde)- |
| | | | | (Pmc) | (Boc) | | | | | Rink Resin |
| 2(24) | Fmoc-Ala | Asp | Asp | Asp | Lys | Ile | Phe | Ser | Ile | Lys(ivDde)- |
| | | (tBu) | (tBu) | (tBu) | (Boc) | | | (OtBu) | | Rink Resin |
| 2(25) | Fmoc-Ala | Leu | Phe | Gly | Phe | Leu | Ala | Arg | Asn | Lys(ivDde)- |
| | | | | | | | | (Pmc) | (Trt) | Rink Resin |
| 2(26) | Fmoc-Ala | Asp | Phe | Leu | Phe | Glu | Gly | Arg | Ala | Lys(ivDde)- |
| | | (tBu) | | | | | | (Pmc) | | Rink Resin |
| 2(27) | Fmoc-Ala | Ile | Phe | Asp | Ser | Val | Leu | Arg | Phe | Lys(ivDde)- |
| | | | | (tBu) | (OtBu) | | | (Pmc) | | Rink Resin |
| 2(28) | Fmoc-Ala | Asn | Ile | Leu | Ala | Pro | Phe | Arg | Phe | Lys(ivDde)- |
| | | (Trt) | | | | | | (Pmc) | | Rink Resin |
| 2(29) | Fmoc-Ala | Ala | Phe | Gly | Ala | Phe | Lys | Arg | Ala | Lys(ivDde)- |
| | | | | | | | (Boc) | (Pmc) | | Rink Resin |
| 2(30) | Fmoc-Ala | Ala | Phe | Leu | Phe | Lys | Gln | Arg | Lys | Lys(ivDde)- |
| | | | | | | (Boc) | (Trt) | (Pmc) | | Rink Resin |
| 2(31) | Fmoc-Ala | Gly | Phe | Phe | Asp | Ala | Pro | Arg | Phe | Lys(ivDde)- |
| | | | | | (tBu) | | | (Pmc) | | Rink Resin |
| 2(32) | Fmoc-Ala | Val | Leu | Asp | Phe | Gly | Pro | Arg | Phe | Lys(ivDde)- |
| | | | | (tBu) | | | | (Pmc) | | Rink Resin |
| 2(33) | Fmoc-Ala | Asp | Phe | Leu | Phe | Ala | Pro | Lys | Phe | Lys(ivDde)- |
| | | (tBu) | | | | | | (Boc) | | Rink Resin |
| 2(34) | Fmoc-Ala | Ser | Phe | Asp(tBu) | Ala | Pro | Phe | Lys(Boc) | Ile | Lys(ivDde)- |
| | | | | | | | | | | Rink Resin |
| 2(35) | Fmoc-Ala | Gly | Phe | Phe | Gly | Val | Arg(Pmc) | Lys(Boc) | Phe | Lys(ivDde)- |
| | | | | | | | | | | Rink Resin |
| 2(36) | Fmoc-Ala | Ile | Phe | Asp | Asp | Asp | Asp | Lys | Ile | Lys(ivDde)- |
| | | | | (tBu) | (tBu) | (tBu) | (tBu) | (Boc) | | Rink Resin |

Compounds 3(₁₋₃₆₎:

The Fmoc protected peptide resins 2₍₁₋₃₆₎ (0.06 mmol) were transferred into 4 ml Alltech tubes and fitted in two batches onto a 24-position SPE Vacuum tank³. Each resin sample was treated with a 20% Piperidine solution in DMF (2 ml) for (2×10 min) and then washed with DMF (6×2 ml). The freshly deprotected resins were then treated with Fmoc-Gly-OH (0.027 g, 0.09 mmol) and Fmoc-(D2)Gly-OH (0.027 g, 0.09 mmol), PyBOP (0.094 g, 0.18 mmol), and HOBt (0.024 g, 0.18 mmol) in a mixture of DMF (1 ml) and DCM (0.5 ml). Finally, Hünig's base (0.047 g, 0.36 mmol) was added and the reactions allowed to proceed at room temperature for 16 h with intermittent manual agitation. The resins were then washed successively with DMF (6×2 ml) and dichloromethane (4×2 ml) to give compounds 3(1-36) (ca 0.350 g, 0.06 mmol) as yellow solids. Kaiser test was negative in each case. 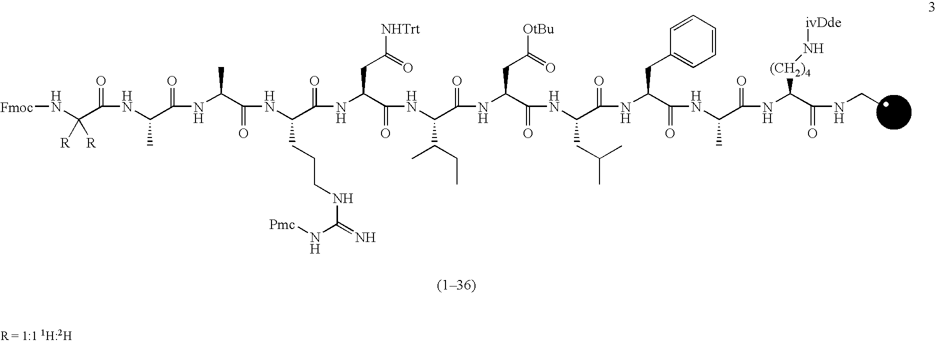
Compounds 4₍₁₋₃₆₎:

The Fmoc protected peptide resins 3₍₁₋₃₆₎ (0.06 mmol) were treated with a 20% Piperidine solution in DMF (2 ml) for (2×10 min) and then washed with DMF (6×2 ml). A 20% solution of acetic anhydride in DMF (2 ml) was then added to each resin sample and the reaction allowed to proceed for 4 h whilst resin samples were manually agitated intermittently. The resins were then washed successively with DMF (6×2 ml) and dichloromethane (4×2 ml) to give compounds 4₍₁₋₃₆₎ (ca 0.310 g, 0.06 mmol) as yellow solids. Kaiser test was negative in each case. 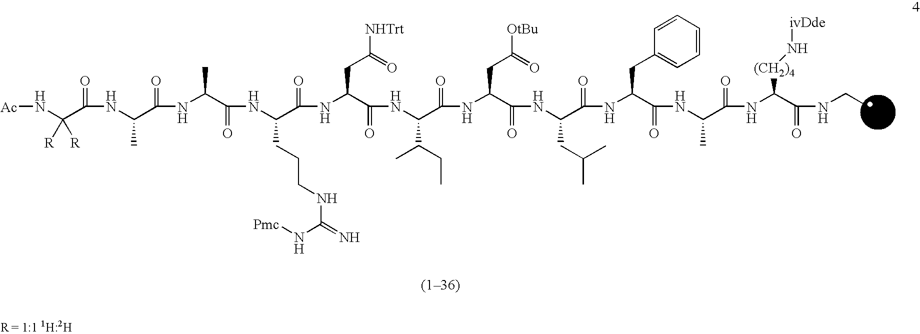
Compounds 5₍₁₋₃₆₎:

The iv(Dde) protected peptide resins 4₍₁₋₃₆₎ (0.06 mmol) were treated with a 20% solution of Hydrazine in DMF (2 ml) for (2×20 min). The resins were then washed successively with DMF (6×2 ml) and dichloromethane (4×2 ml) to give amine resins 5₍₁₋₃₆₎ (ca 0.300 g, 0.06 mmol) as yellow solids. Kaiser test was positive in all cases. 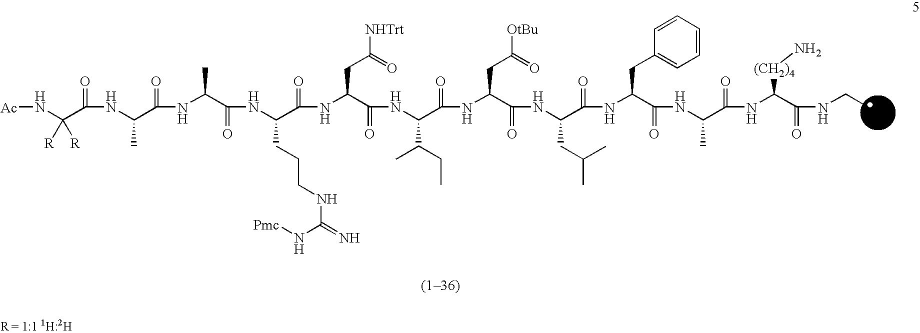
Compounds 6₍₁₋₃₆₎:

The amine resins 5₍₁₋₃₆₎ (0.06 mmol) were treated with a solution of Acetic anhydride (0.2 ml) and Acetic anhydride-d₆ (0.2 ml) in DMF (1.8 ml) and the reaction was allowed to proceed for 4 h with intermittent manual shaking. The resins were then washed successively with DMF (6×2 ml), dichloromethane (4×2 ml) and ether (2×2 ml) then dried in vacuo to give peptide resins 6₍₁₋₃₆₎ (ca 0.400 g, 0.06 mmol) as yellow solids. Kaiser test was negative in each case. 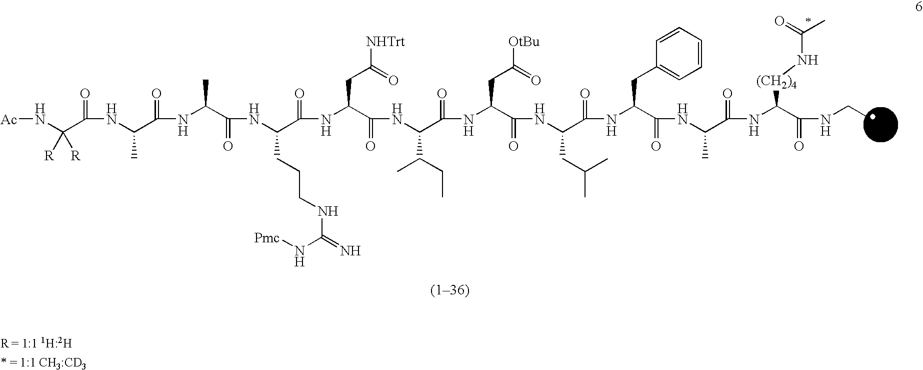
Compounds 7₍₁₋₃₆₎:

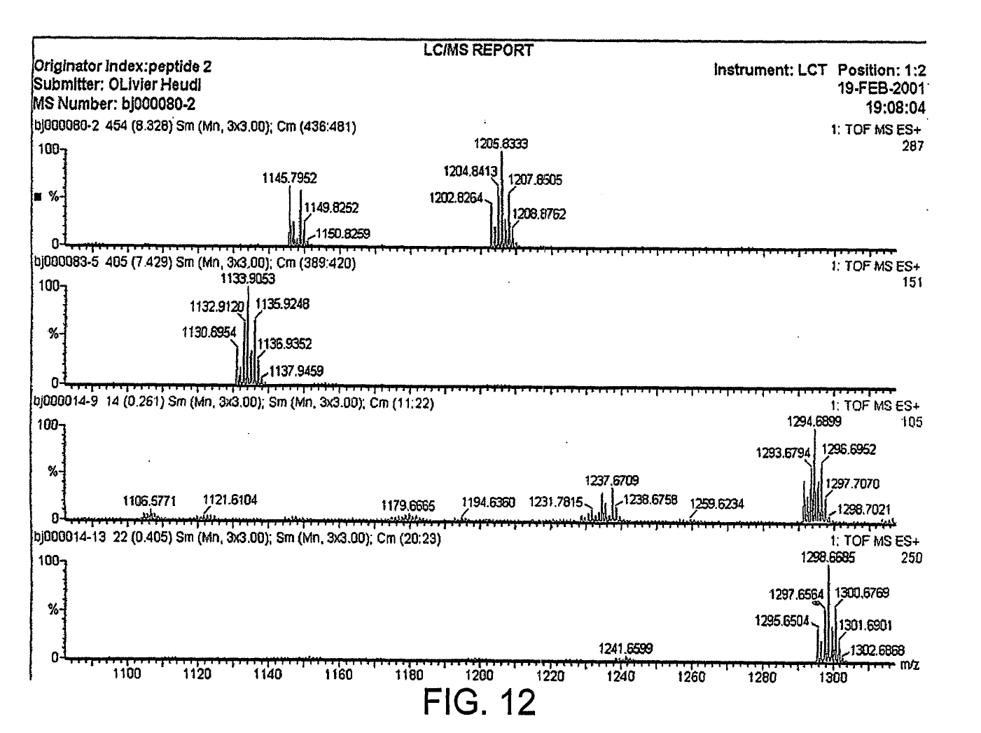

The peptide resins 6₍₁₋₃₆₎ (0.06 mmol) were swollen in dichloromethane for 10 min and the solvents drained under vacuum. Trifluoroacetic acid (1.5 ml) was added to each resin sample and the cleavage reaction allowed to proceed for 2 h. The resins were drained and then further washed with dichloromethane (6×1 ml). The combined filtrates were concentrated under a stream of nitrogen then dried in vacuo to give DiMaS tagged peptides 7₍₁₋₃₆₎ (ca 0.5 mg) as white solids or pale brown oils. The expected multiplet pattern was observed in all 36 cases (Table 3) MS Analysis FIG. 12).

TABLE 3

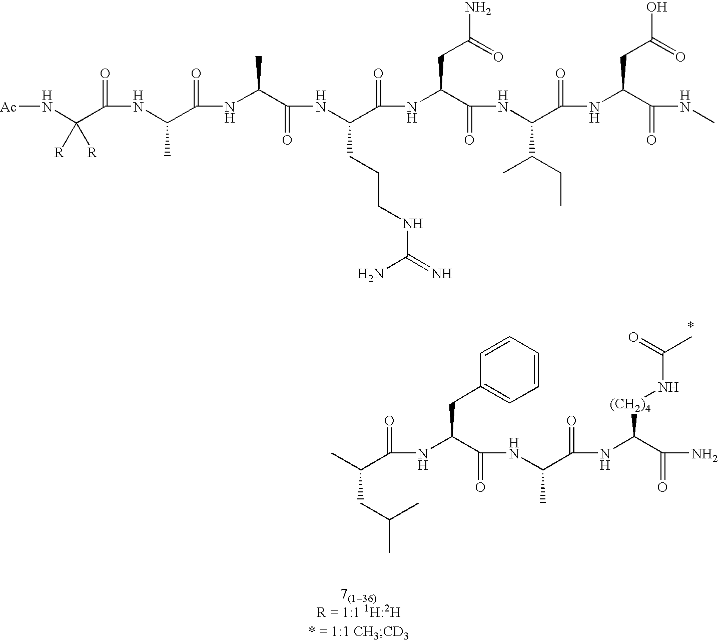

Characterisation of Compounds 7₍₁₋₃₆₎:

| no | | Av Mw |
|---|---|---|
| 7(1) | Ac-Gly* Ala Ala Arg Asn Ile Asp Leu Phe Ala Lys(Ac*)-NH₂ | 1259 |
| 7(2) | Ac-Gly* Ala Gly Arg Ala Asn Ile Asp Phe Ala Lys(Ac*)-NH₂ | 1203 |
| 7(3) | Ac-Gly* Ala Leu Arg Phe Ala Asn Ile Phe Ala Lys(Ac*)-NH₂ | 1291 |
| 7(4) | Ac-Gly* Ala Phe Arg Phe Ala Ala Asn Ile Ala Lys(Ac*)-NH₂ | 1249 |
| 7(5) | Ac-Gly* Ala Lys Arg Ala Gly Ala Ala Phe Ala Lys(Ac*)-NH₂ | 1131 |
| 7(6) | Ac-Gly* Ala Gln Arg Lys Val Gly Ala Phe Ala Lys(Ac*)-NH₂ | 1216 |
| 7(7) | Ac-Gly* Ala Pro Arg Ser Asp Val Gly Phe Ala Lys(Ac*)-NH₂ | 1188 |
| 7(8) | Ac-Gly* Ala Pro Arg Phe Ser Asp Val Leu Ala Lys(Ac*)-NH₂ | 1244 |
| 7(9) | Ac-Gly* Ala Pro Lys Ile Gly Ser Asp Phe Ala Lys(Ac*)-NH₂ | 1174 |
| 7(10) | Ac-Gly* Ala Phe Lys Ile Ile Gly Ser Phe Ala Lys(Ac*)-NH₂ | 1222 |
| 7(11) | Ac-Gly* Ala Arg Lys Phe Ile Ile Ile Phe Ala Lys(Ac*)-NH₂ | 1347 |
| 7(12) | Ac-Gly* Ala Asp Lys Ile Phe Ser Gly Phe Ala Lys(Ac*)-NH₂ | 1224 |
| 7(13) | Ac-Gly* Ala Phe Leu Ala Arg Asn Ile Asp Leu Lys(Ac*)-NH₂ | 1301 |
| 7(14) | Ac-Gly* Ala Phe Glu Gly Arg Ala Asn Ile Asp Lys(Ac*)-NH₂ | 1261 |
| 7(15) | Ac-Gly* Ala Ser Val Leu Arg Phe Ala Asn Ile Lys(Ac*)-NH₂ | 1259 |
| 7(16) | Ac-Gly* Ala Ala Pro Phe Arg Phe Ala Ala Asn Lys(Ac*)-NH₂ | 1233 |
| 7(17) | Ac-Gly* Ala Ala Phe Lys Arg Ala Gly Ala Ala Lys(Ac*)-NH₂ | 1131 |
| 7(18) | Ac-Gly* Ala Phe Lys Gln Arg Lys Val Gly Ala Lys(Ac*)-NH₂ | 1273 |
| 7(19) | Ac-Gly* Ala Asp Ala Pro Arg Ser Asp Val Gly Lys(Ac*)-NH₂ | 1156 |
| 7(20) | Ac-Gly* Ala Phe Gly Pro Arg Phe Ser Asp Val Lys(Ac*)-NH₂ | 1264 |
| 7(21) | Ac-Gly* Ala Phe Ala Pro Lys Ile Gly Ser Asp Lys(Ac*)-NH₂ | 1174 |
| 7(22) | Ac-Gly* Ala Ala Pro Phe Lys Ile Ile Gly Ser Lys(Ac*)-NH₂ | 1172 |
| 7(23) | Ac-Gly* Ala Gly Val Arg Lys Phe Ile Ile Gly Lys(Ac*)-NH₂ | 1229 |
| 7(24) | Ac-Gly* Ala Asp Asp Asp Lys Ile Phe Ser Ile Lys(Ac*)-NH₂ | 1292 |
| 7(25) | Ac-Gly* Ala Leu Phe Gly Phe Leu Ala Arg Asn Lys(Ac*)-NH₂ | 1277 |
| 7(26) | Ac-Gly* Ala Asp Phe Leu Phe Glu Gly Arg Ala Lys(Ac*)-NH₂ | 1294 |
| 7(27) | Ac-Gly* Ala Ile Phe Asp Ser Val Leu Arg Phe Lys(Ac*)-NH₂ | 1336 |
| 7(28) | Ac-Gly* Ala Asa Ile Leu Ala Pro Phe Arg Phe Lys(Ac*)-NH₂ | 1317 |
| 7(29) | Ac-Gly* Ala Ala Phe Gly Ala Phe Lys Arg Ala Lys(Ac*)-NH₂ | 1217 |
| 7(30) | Ac-Gly* Ala Ala Phe Leu Phe Lys Gln Arg Lys Lys(Ac*)-NH₂ | 1377 |
| 7(31) | Ac-Gly* Ala Gly Phe Phe Asp Ala Pro Arg Phe Lys(Ac*)-NH₂ | 1296 |
| 7(32) | Ac-Gly* Ala Val Leu Asp Phe Gly Pro Arg Phe Lys(Ac*)-NH₂ | 1290 |
| 7(33) | Ac-Gly* Ala Asp Phe Leu Phe Ala Pro Lys Phe Lys(Ac*)-NH₂ | 1324 |
| 7(34) | Ac-Gly* Ala Ser Phe Asp Ala Pro Phe Lys Ile Lys(Ac*)-NH₂ | 1264 |
| 7(35) | Ac-Gly* Ala Gly Phe Phe Gly Val Arg Lys Phe Lys(Ac*)-NH₂ | 1297 |

### Example 9

### Incubation of Compounds 7(2) and 7(13) with Trypsin

Compounds 7(2) and 7(13) (10 μl in Acetonitrile) were treated with a solution of buffer (50 mM HEPES, 150 mM NaCl and 1 μM Zn²⁺, 35 μl), and finally Trypsin was added (5 μl, 2.5 μM). The reaction was allowed to take place at room temperature for 9 h and an aliquot of solution was removed for LC-MS analysis every 2 h.

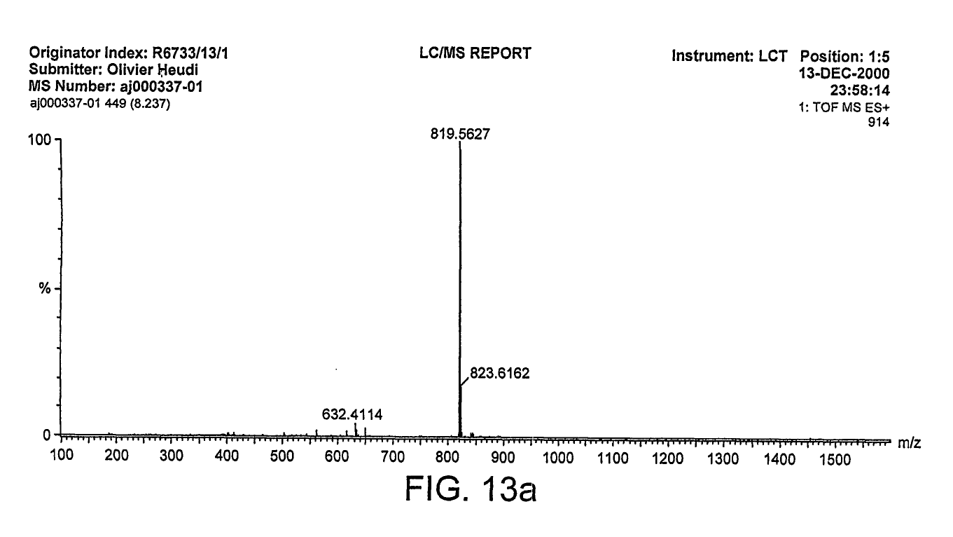

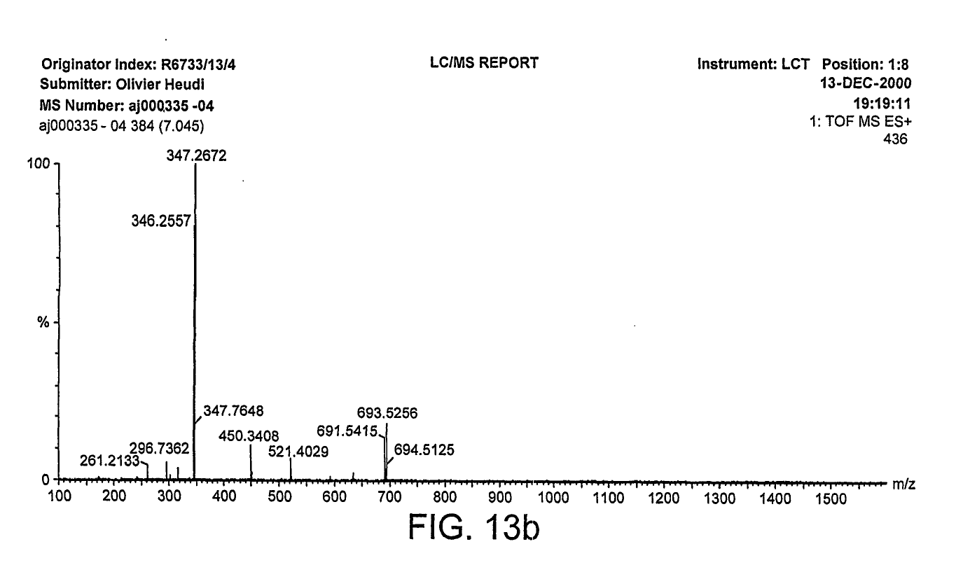

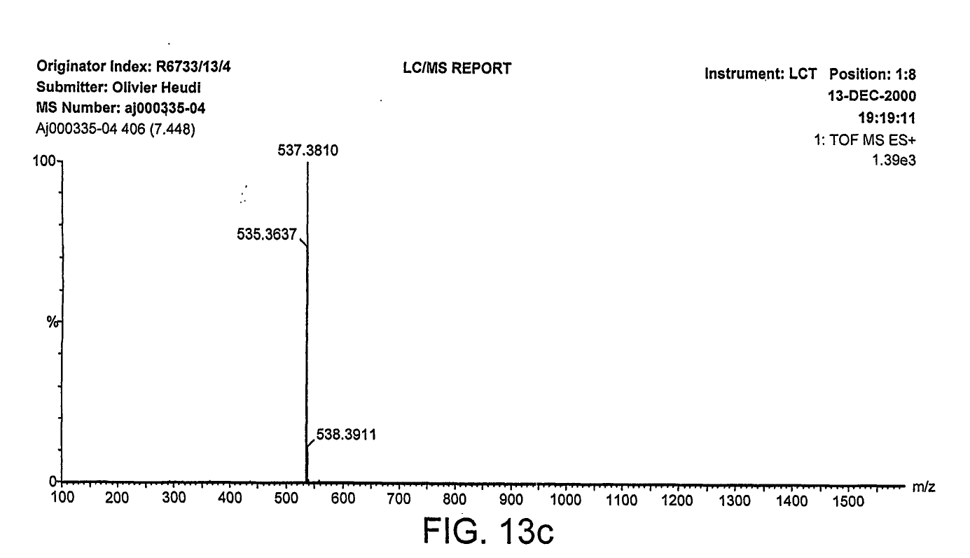

Incubation of peptides 7(2) and 7(13) with Trypsin as described above gave the expected peptide fragments corresponding to cleavage after the arginine residue. Both sets of doublets were observed in most cases (MS Analysis shown in FIG. 13).

### Example 10

### Preparation of Pools

Compounds 8₍₁₋₄₎:

The linear peptide was assembled on an ABI 433A automated peptide synthesizer. The Fmoc protected Rink amide resin (0.12 g, 0.06 mmol) was coupled successively with Fmoc-Lys(ivDde)-OH, Fmoc-Phe-OH, Fmoc-Asp(OtBu)-OH, Fmoc-Ile-OH, Fmoc-Asn(Trt)-OH, Fmoc-Ala-OH and Fmoc-Arg(Pmc)-OH, using the HBTU (1.0 mmol) activation cycle and extended coupling time (2 h). The introduction of a mixture of (Fmoc-Cha-OH:Fmoc-Pro-OH:Fmoc-Leu-OH:Fmoc-Glu-OH) (1:1:1:1), (1.00 mmol total) was performed online. This was followed by coupling with Fmoc-Ala-OH, (1.0 mmol), then coupled with a mixture of [Fmoc-(H₂)Gly-OH:Fmoc-(D₂)Gly-OH) (1:1), (1 mmol total). Finally the assembled peptide resin was transferred into an Alltech tube and was treated with a solution of Acetic anhydride (0.2 ml) and Acetic anhydride-d₆ (0.2 ml) in DMF (1.8 ml) and the reaction was allowed to proceed for 4 h with intermittent manual shaking. The resin was then washed successively with DMF (6×2 ml), dichloromethane (4×2 ml) and ether (2×2 ml) then dried in vacuo to give peptide resin 8₍₁₋₄₎ (ca 0.400 g, 0.06 mmol) as a yellow solid. Kaiser test was negative.

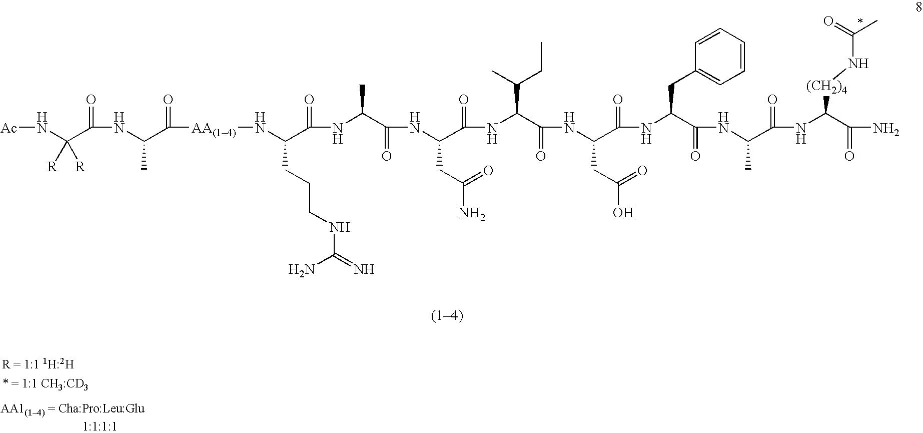

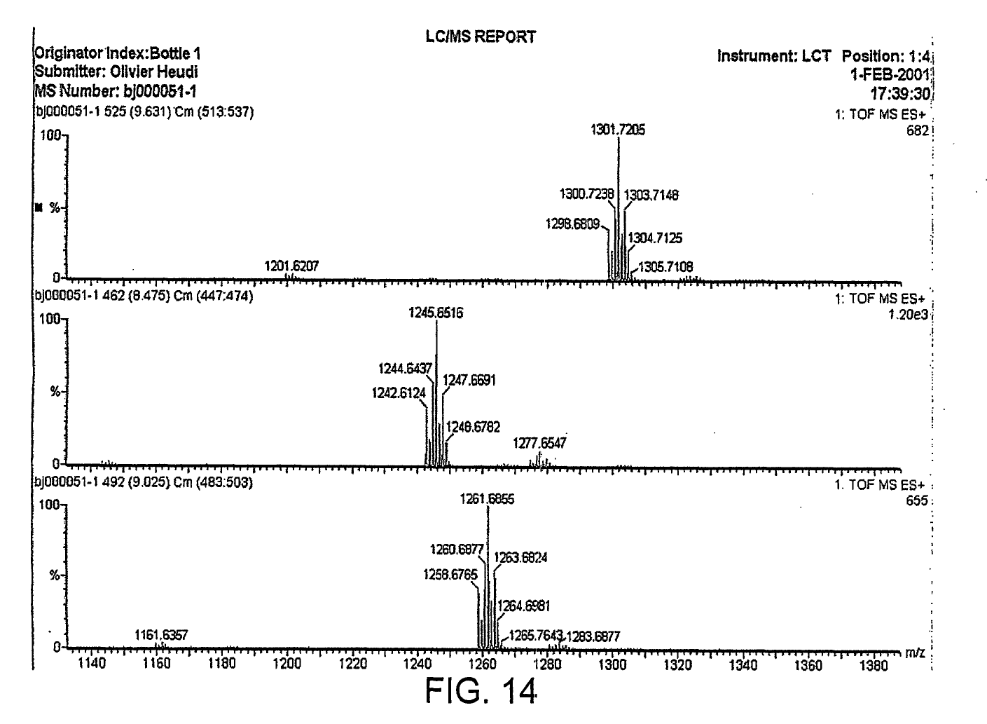

The expected multiplet pattern was observed in all four cases (Table 4) MS Analysis bj000051-1 (FIG. 14).

TABLE 4
Characterisation of Peptides 8₍₁₋₄₎:

| | | | | | | | | | | | | MW |
|---|---|---|---|---|---|---|---|---|---|---|---|---|
| 8(1) | Ac-Gly* | Ala | Cha | Arg | Ala | Asn | Ile | Asp | Phe | Ala | Lys(Ac*)-NH2 | 1299 |
| 8(2) | Ac-Gly* | Ala | Pro | Arg | Ala | Asn | Ile | Asp | Phe | Ala | Lys(Ac*)-NH2 | 1243 |
| 8(3) | Ac-Gly* | Ala | Leu | Arg | Ala | Asn | Ile | Asp | Phe | Ala | Lys(Ac*)-NH2 | 1259 |
| 8(4) | Ac-GIy* | Ala | Glu | Arg | Ala | Asn | Ile | Asp | Phe | Ala | Lys(Ac*)-NH2 | 1275 |

Compounds 8₍₅₋₄₎:

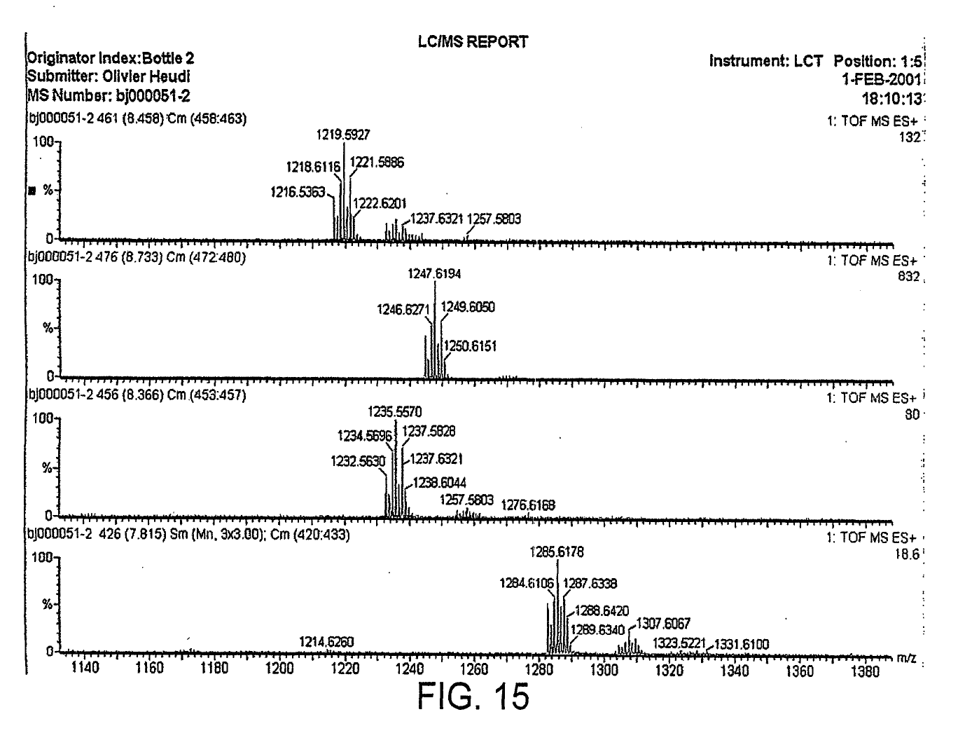

Peptides 8₍₅₋₈₎ were prepared in an analogous fashion to peptides 8₍₁₋₄₎. The expected MS pattern was observed in all four cases. The expected multiplet pattern was observed in all four cases (Table 5) MS Analysis bj000051-2 (FIG. 15).

TABLE 5

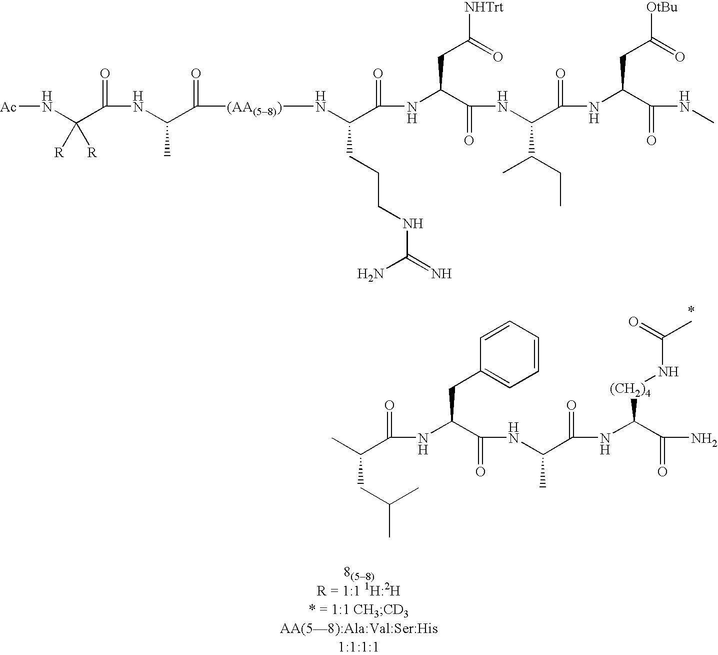

Characterisation of Peptides 8₍₅₋₈₎:

| | | MW |
|---|---|---|
| 8(5) | Ac-Gly* Ala Ala Arg Ala Asn Ile Asp Phe Ala Lys(Ac*)-NH₂ | 1217 |
| 8(6) | Ac-Gly* Ala Val Arg Ala Asn Ile Asp Phe Ala Lys(Ac*)-NH₂ | 1245 |
| 8(7) | Ac-Gly* Ala Ser Arg Ala Asn Ile Asp Phe Ala Lys(Ac*)-NH₂ | 1233 |
| 8(8) | Ac-Gly* Ala His Arg Ala Asn Ile Asp Phe Ala Lys(Ac*)-NH₂ | 1283 |

### Example 11

### Incubation with Trypsin

Incubation of Compounds 8₍₁₋₄₎ with Trypsin

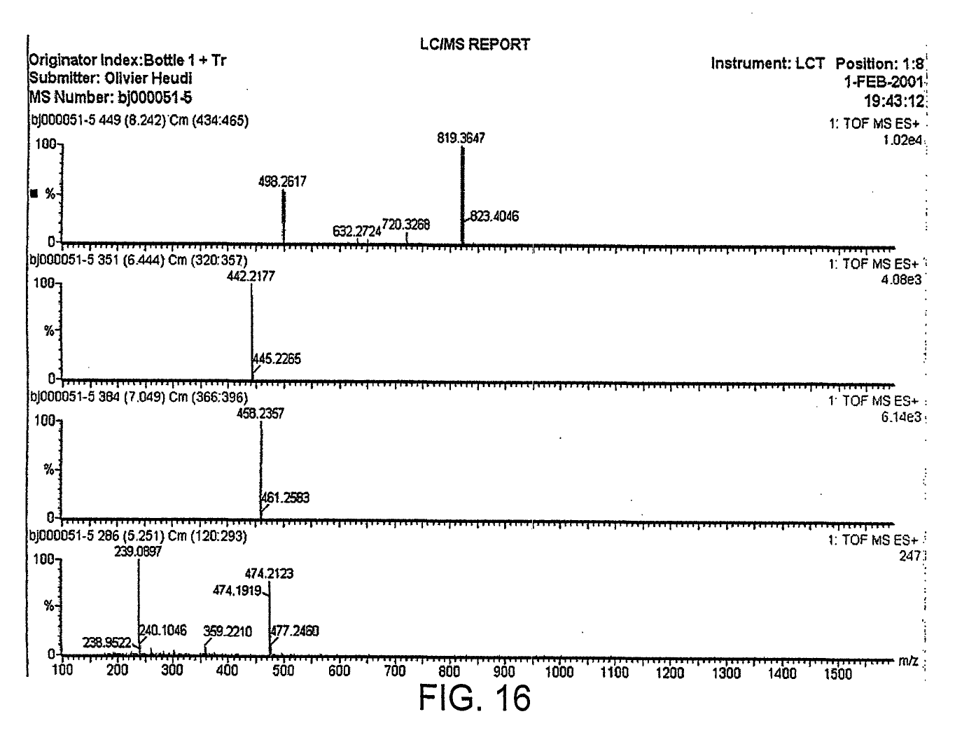

The peptide mixture 8₍₁₋₄₎ (30 μl in Acetonitrile) was treated with a solution of buffer (50 mM HEPES, 150 mM NaCl and 1 μM Zn²⁺, 100 μl), and finally Trypsin was added (10 μl, 5 μM). The reaction was allowed to take place at room temperature for 9 h and an aliquot of solution was removed for LC-MS analysis every 2 h. Incubation with Trypsin gave the expected peptide fragments corresponding to cleavage after the arginine residue. Both sets of doublets observed in most cases (Table 6) MS Analysis bj000051-5 (FIG. 16).

TABLE 6
Results of incubation of Compounds 8₍₁₋₄₎ with Trypsin:

| | Mw | LHS | RHS |
|---|---|---|---|
| 8(1) | 1299 | 498, 500 | 819, 822 |
| 8(2) | 1243 | 442, 444 | 819, 822 |
| 8(3) | 1259 | 458, 460 | 819, 822 |
| 8(4) | 1275 | | 819, 822 |

Incubation of Compounds 8₍₅₋₈₎ with Trypsin:

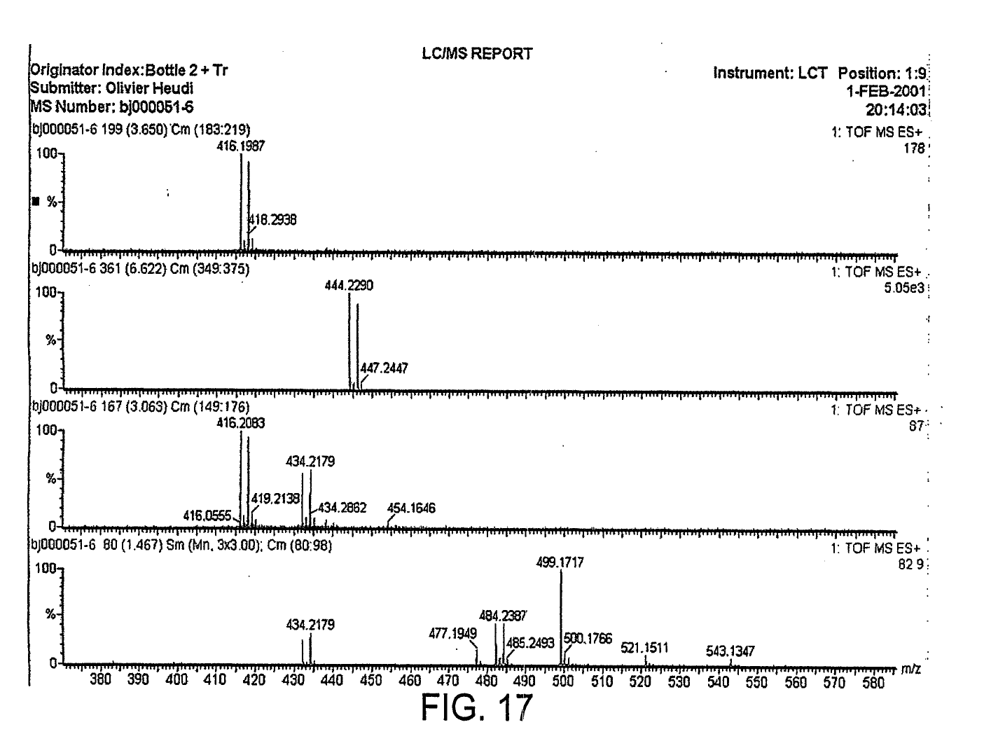

The peptide mixture 8₍₅₋₈₎ was incubated with Trypsin as described above to give the expected peptide fragments corresponding to cleavage after the arginine residue. Both sets of doublets observed in most cases (Table 7) MS Analysis bj000051-6 (FIG. 17).

TABLE 7
Results of incubation of Compounds 8₍₅₋₈₎ with Trypsin

| | Mw | LHS | RHS |
|---|---|---|---|
| 8(5) | 1217 | 416, 418 | 819, 822 |
| 8(6) | 1245 | 444, 446 | 819, 822 |
| 8(7) | 1233 | 432, 434 | 819, 822 |
| 8(8) | 1283 | (—) | 819, 822 |

### Example 12

### Peptide Sequencing

The sequence or primary structure of a peptide may be determined by cleaving or fragmentation of the peptide and subsequent analysis by mass spectrometry.

In the case of a peptide which has been differentially isotopically labelled at the N-terminus amino acid, fragmentation in the C— to N-direction retains the peak split caused by the isotopic label giving rise to doublets, while the reverse fragmentation in the N— to C-direction produces only single mass peaks (FIG. 18).

This data can then be used to determine the amino acid sequence of the peptide. The difference in sizes of the fragments will indicate the sizes of the amino acids which have been removed, and this data may be used to determine the primary sequence of the original molecule.

References:

1. Kaiser, E.; Colescott, R. L.; Bossinger, C. D.; Cook, P. I. Anal. Biochem., 1970, 34, 595.
2. Atherton, E.; Sheppard, R. C. In ‘Solid Phase Peptide Synthesis’, 1989, Practical Approach Series, IRL Press, Oxford.
3. Available from Whatman
   Abbreviations
AcOH=Acetic Acid
Boc=tert-Butyloxycarbonyl
DIPEA=Diispropylethylamine
DMAP=Dimethylaminopyridine
DMF=Dimethylformamide
Fmoc=Fluorenylmethoxycarbonyl
Fmoc-Lys(ivDde)-OH═N-α-Fmoc-N-ε-I-(4,4-dimethyl-2,6-dioxocyclohex-1-ylidene)-3-methylbutyl-L-lysine. 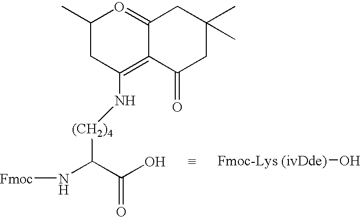
HBTU=2-(1H-Benzotriazole-1-yl)-1,1,3,3-tetramethyluronium hexafluorophosphate
HOBt=Hydroxybenzotriazole
NMP=N-Methyl pyrrolidone
MS Mass Spectroscopy
PG=Protecting group
PyBOP=Benzotriazole-1-yl-oxy-tris-pyrrolidino-phosphonium hexafluorophosphate
Rink Amide Resin=4-(2′,4′-Dimethoxyphenyl-Fmoc-aminomethyl) 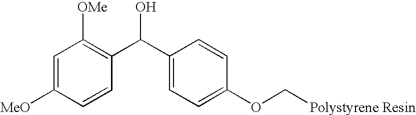
TFA=Trifluoroacetic Acid
Bzl=Benzyl
Cbz=Carbobenzyloxy
DCM=Dichloromethane
Fmoc=Fluorenylmethoxycarbonyl
TFA=Trifluroacetic Acid
TIPS=Triisopropylsilane

1. A method of analyzing cleavage of a polymer, the method comprising:
(a) providing a sample of said polymer, a portion of said polymer molecules having been labeled at a position on one side of a potential cleavage site with a first isotopic label and a portion of said polymer molecules having been labeled at a position on the opposite side of the potential cleavage site with a second isotopic label;
(b) incubating said sample under conditions suitable for cleavage at said potential cleavage site, and
(c) analyzing the mass(es) of any cleaved fragments by mass spectrometry and thereby determining whether and/or where cleavage has taken place.

2. A method according to claim 1 wherein the mass change caused by the presence of the first isotopic label compared with the mass in the presence of the normal element(s) at that position in different to the mass change caused by the presence of the second isotopic label compared with the mass in the presence of the normal element(s) at that position.

3. A method according to claim 1 wherein
(a) 50% of the polymer molecules in said sample comprise said first isotopic label, and/or
(b) 50% of the polymer molecules in said sample comprise said second isotopic label.

4. A method according to claim 3 wherein said sample comprises a portion of polymer molecules which comprise both said first isotopic label and said second isotopic label, a portion of polymer molecules which comprise said first isotopic label but not said second isotopic label, a portion of polymer molecules which comprise said second isotopic label but not said first isotopic label and a portion of said polymer labels which comprise neither said first nor second isotopic labels.

5. A method according to claim 1 wherein the first and second isotopic labels comprise different chemical isotopes.

6. A method according to claim 1 wherein the first and second isotopic labels comprise the same chemical isotope and wherein each of said first and second isotopic labels comprise a different number of atoms of said chemical isotope.

7. A method according to claim 1 wherein said isotopic labels comprise ²H, ¹³C, ¹⁸O, ¹⁵N and/or ⁸¹Br.

8. A method according to claim 1 wherein said polymer is a linear polymer.

9. A method according to claim 1 wherein said polymer comprises a peptide or protein.

10. A method according to claim 9 wherein said first and second isotopic labels are located in the terminal amino acids of the peptide or protein.

11. A method according to claim 1 wherein said polymer further comprises a moiety which allows for separation of the molecule attached to the moiety from other material.

12. A method according to claim 1 wherein the sample or library is incubated with an enzyme under conditions suitable for cleavage of a substrate.

13. A method according to claim 12 wherein said enzyme is a protease.

14. A method according to claim 1 wherein the mass(es) of the cleaved fragments are used to calculate the location of the cleavage site.

15. A method according to claim 1, the method further comprising the step of quantifying the amount of the polymer or cleaved fragment(s) present after cleavage.

16. A method according to claim 1 wherein said polymer further comprises a detectable non-isotopic label on one side of the potential cleavage site and an inhibitor for said label on the opposite side of the potential cleavage site, the method comprising the additional step of detecting said detectable non-isotopic label, wherein the detection of said label indicates that said polymer has been cleaved.

17. A method according to claim 16 wherein said detectable label is a fluorescent group and said inhibitor is a quencher for said fluorescent group.

18. A method according to claim 1 wherein said polymer is attached to a solid support.

19. A method according to claim 18 wherein the polymer is attached via a releasable linker molecule.

20. A method according to claim 18 wherein said solid support is a resin bead.

21. A method according to claim 1 further comprising determining the structure or sequence of one or both of the cleaved fragments.

22. A method according to claim 21 wherein the cleaved fragment(s) are further cleaved and the masses of these further fragments determined using mass spectrometry.

23. A kit for screening a library of polymers for cleavage, comprising two or more polymers a portion of said polymers having been labeled at a position on one side of a potential cleavage site with a first isotopic label and a portion of said polymers having been labeled at a position on the opposite side of the potential cleavage site with a second isotopic label.

24. A kit according to claim 23 wherein said polymers are supplied in solid form or in solutions.

25. A kit according to claim 23 wherein said polymers are supplied on a solid support and attached via a releasable linker molecule.

26. A kit according to claim 25 further comprising a linker cleaving reagent.

27. A kit for the preparation of a polymer as described in claim 1, comprising a set of chemical monomers which comprises monomers which have been isotopically labeled and monomers which have no isotopic label, in a form suitable for polymer synthesis.

28. A kit according to claim 27 further comprising means for adding an isotopic label to an unlabelled monomer.

29. A kit according to claim 27 wherein said set of monomers comprises:
(a) a number of isotopically labeled amino acids and a number of amino acids which have no isotopic label; or
(b) a number of isotopically labeled nucleotides and a number of nucleotides which have no isotopic label.

30. A method of screening a library of polymers for cleavage, the method comprising:
(a) providing a library comprising two or more polymers, a portion of said polymers having been labeled at a position on one side of a potential cleavage site and a portion of said polymers having been labeled at a position on the opposite side of the potential cleavage site with a second isotopic label,
(b) incubating said library of polymers under conditions suitable for cleavage, and
(c) analyzing the mass(es) of any cleaved fragments by mass spectrometry and thereby determining whether and/or where cleavage has taken place.
